SEQUENCE LISTING (15 sequences)

SEQ ID NO: 1 - DNA, 31 bp, Ebola virus
ctactgtatttcataagaagagagttgaacc

SEQ ID NO: 2 - DNA, 30 bp, Ebola virus
attagtaggagtaattccctatcagttaaa

SEQ ID NO: 3 - DNA, 48 bp, Ebola virus
  misc_feature (32)..(32): tetrahydrofuran (THF)
atatgtccgaccttgaaaaaaggatttttgtntgacagtagtttttgc

SEQ ID NO: 4 - DNA, 31 bp, Ebola virus; identical to SEQ ID NO: 1

SEQ ID NO: 5 - DNA, 30 bp, Ebola virus; identical to SEQ ID NO: 2

SEQ ID NO: 6 - DNA, 47 bp, Ebola virus
  misc_feature (31)..(31): tetrahydrofuran (THF)
aatccttttttcaaggtcggacatatgtctntaggtgctggaggaac

SEQ ID NO: 7 - DNA, 32 bp, Ebola virus
ctactgagtccagtatagagtcagaaatagta

SEQ ID NO: 8 - DNA, 30 bp, Ebola virus
ctgagttgttaagaataatctcaatttggt

SEQ ID NO: 9 - DNA, 48 bp, Ebola virus
  misc_feature (32)..(32): tetrahydrofuran (THF)
aatgactactcctaggatgcttctacctgttntgtcaaaattccataa

SEQ ID NO: 10 - DNA, 32 bp, Ebola virus
gacgacaatcctggccatcaagatgatgatcc

SEQ ID NO: 11 - DNA, 31 bp, Ebola virus
cgtcctcgtctagatcgaataggaccaagtc

SEQ ID NO: 12 - DNA, 48 bp, Ebola virus
  misc_feature (30)..(30): tetrahydrofuran (THF)
  misc_feature (32)..(32): n is a, c, g, or t
gatgatggaagctacggcgaataccagagttnctcggaaaacggcatg

SEQ ID NO: 13 - DNA, 47 bp, Ebola virus
atatgtccgaccttgaaaaaaggatttttgttgacagtagtttttgc

SEQ ID NO: 14 - DNA, 46 bp, Ebola virus
aatccttttttcaaggtcggacatatgtcttaggtgctggaggaac

SEQ ID NO: 15 - DNA, 47 bp, Ebola virus
aatgactactcctaggatgcttctacctgtttgtcaaaattccataa

(19) United States
(12) Patent Application Publication
(10) Pub. No.: US 2018/0245144 A1
(43) Pub. Date: Aug. 30, 2018

(54) PAPER DEVICE FOR GENETIC DIAGNOSIS

(71) Applicants: ECOLE SUPÉRIEURE DE PHYSIQUE ET DE CHIMIE INDUSTRIELLES DE LA VILLE DE PARIS, PARIS (FR); CENTRE NATIONAL DE LA RECHERCHE SCIENTIFIQUE, PARIS CEDEX 16 (FR); INSTITUT PASTEUR, PARIS CEDEX 15 (FR)

(72) Inventors: Laura MAGRO, PARIS (FR); Pierre LAFAYE, MALAKOFF (FR); Fabrice MONTI, SAULX LES CHARTREUX (FR); Patrick TABELING, PARIS (FR); Jean-Claude MANUGUERRA, PARIS (FR); Jessica VANHOMWEGEN, PARIS (FR); Béatrice JACQUELIN, PARIS (FR); Anavaj SAKUNTHABHAI, PARIS (FR)

(21) Appl. No.: 15/550,716

(22) PCT Filed: Feb. 12, 2016
(86) PCT No.: PCT/EP2016/053077

§ 371 Date: Aug. 11, 2017

(30) Foreign Application Priority Data
Feb. 13, 2015 (FR) 1551189
Sep. 15, 2015 (FR) 1558657

(51) Int. Cl. C12Q 1/6844; C12M 3/06; B01L 3/00; C12Q 1/70

(52) U.S. Cl. CPC C12Q 1/6844; C12M 23/16; B01L 3/5027; C12Q 1/701; B01L 2300/0816; B01L 2400/0406; B01L 2300/126; B01L 2300/1805; B01L 2300/161; B01L 3/502707

(57) ABSTRACT

A device for diagnosis by DNA comprises: a porous substrate comprising at least: an area for depositing a sample, the sample comprising at least one target compound; a plurality of channels positioned in the thickness of the porous substrate; an area, referred to as the “diagnostic area”, comprising at least one reactive compound suitable for reacting with a so-called target compound; an area for depositing a carrier vector suitable for being transported by capillarity into all of areas and channels: each of the areas being linked to the others by at least one element chosen from a channel and an area, a means for locally conditioning the temperature of the diagnostic area.

The invention relates to devices for diagnosis by DNA amplification. It applies, in particular, to the diagnosis of microorganisms, using a biological sample such as drops of blood, of urine, of saliva and of sweat.

In the medical diagnosis field, there are, inter alia, two families of biological tests.

Firstly, the immunoassays, among which are the “immunoenzymatic” assays, abbreviated to ELISA (for “enzyme-linked immunosorbent assay”), having the objective of detecting the presence of an antibody or of an antigen in a sample. The formation and the spatial localization of the antibody-antigen complex is detectable by several techniques: grafting of colored or fluorescent species, nanoparticles and enzymes for example. The signal which characterizes the presence of the species being sought can be detected by eye or by spectroscopy. The tests of strip, dipstick or lateral flow test type are among this family. These point-of-care tests are very suitable for situations in the field where little material is available. However, diagnosis by immunoassays is restricted to detecting species involved in antibody-antigen complexes. For example, in the case of a microorganism, the detection is indirect: the immunoassays make it possible to detect the presence or absence of characteristic proteins or of antibodies produced by the immunocompetent cells of the host infected by the microorganism(s).

Secondly, the DNA amplification tests make it possible to recognize and multiply a specific DNA or RNA sequence. In the case of a microorganism, the DNA amplification test is more direct: the genetic material of the species being sought is recognized. However, these tests require expensive material and in particular know-how to carry them out. These tests do not make it possible to perform point-of-care assays, this being particularly in geographical areas far from laboratories.

For all these reasons, these DNA amplification tests do not make it possible to rapidly and inexpensively carry out diagnoses outside laboratories.

A subject of the present invention is a device for diagnosis by DNA amplification, characterized in that it comprises:

a porous substrate comprising at least:
an area for depositing a sample, said sample comprising at least one target compound;
a plurality of channels positioned in the thickness of said porous substrate;
an area, referred to as the “diagnostic area”, comprising at least one reactive compound suitable for reacting with a said target compound;
an area for depositing a carrier fluid suitable for being transported by capillarity into all of said areas and said channels:

each of said areas being linked to the others by at least one element chosen from a channel and an area,

a means for locally conditioning the temperature of the diagnostic area.

Advantageously, the reactive compounds of said device comprise at least primers, recombinases, polymerases and proteins which bind and maintain the single-standard DNA, said reactive compounds being arranged in the pores of said areas of said porous substrate.

Advantageously, said primers of the device are suitable for detecting the presence of a pathogenic agent by RT-RPA.

Advantageously, said pathogenic agent is the Ebola virus.

Advantageously, at least one said reactive compound is arranged in or on said porous substrate in lyophilized form.

Advantageously, said porous substrate of the device comprises a stack of at least two primary porous substrates, each said primary porous substrate comprising at least one element chosen from a channel, a part of a channel and an area.

Advantageously, said stack of said primary porous substrates of the device is obtained by folding.

Advantageously, the device comprises at least one additional area comprising at least one reactive compound of the diagnostic area linked to the area for depositing a carrier fluid by a succession of at least one element chosen from a channel and an area other than an area for depositing a sample.

Advantageously, a said compound of the device chosen from a reactive compound and a target compound is arranged in or on said porous substrate in lyophilized form.

Advantageously, the device comprises at least one additional area comprising at least one said reactive compound, and at least one target compound, linked to the area for depositing a carrier fluid by a succession of at least one element chosen from a channel and an area other than an area for depositing a sample.

Advantageously, the device comprises a means for locally conditioning the temperature of the area for depositing a sample.

Advantageously, said porous substrate of the device comprises at least one sheet of paper.

Advantageously, said means for locally conditioning the temperature of the device comprises a conducting electrical circuit placed facing said porous substrate.

Advantageously, the device comprises:

a support layer for the conducting electrical circuit and

a leak-tight layer, the leak-tight layer being positioned in a manner inserted between the porous substrate (105) and the support layer.

Advantageously, said conducting electrical circuit of the device is supported by a sheet of paper distinct from said porous substrate.

Advantageously, at least one channel of the device is delimited by regions of said porous substrate which are impregnated with solid wax.

Advantageously, at least one channel of the device is formed by a hydrophilic-hydrophobic contrast in the thickness of said porous substrate.

Advantageously, said means for locally conditioning the temperature of the device is configured so as to fix the temperature of at least one area between 20° C. and 120° C. and preferentially between 30° C. and 40° C.

Advantageously, at least one said reactive compound of the device is suitable for changing color on contact with a said target compound.

Advantageously, at least one said reactive compound of the device is suitable for emitting a fluorescent signal on contact with a said target compound.

Advantageously, said reactive compounds of the device comprise at least one pair of said primers which is selected from the group consisting of SEQ ID NOS.: 1-2; SEQ ID NOS.: 4-5; and SEQ ID NOS.: 7-8.

Advantageously, said reactive compounds of the device comprise at least one nucleotide probe selected from the group consisting of SEQ ID NOS.: 3, 6, 9, 13, 14 and 15.

Advantageously, said reactive compounds of the device comprise at least one composition of oligonucleotides comprising a pair of primers and a probe, selected from the group consisting of a pair of primers of sequence SEQ ID NOS.: 1-2 and a nucleotide probe of sequence SEQ ID NO.: 3; a pair of primers of sequence SEQ ID NOS.: 4-5 and a nucleotide probe of sequence SEQ ID NO.: 6; and a pair of primers of sequence SEQ ID NOS.: 7-8 and a nucleotide probe of sequence SEQ ID NO.: 9.

Another subject of the invention is a process for detecting RNA, comprising at least the steps consisting in:

depositing a said sample on at least one said area for depositing a sample of a said device;

bringing a said carrier fluid into contact with said area for depositing a carrier fluid of said device;

conditioning a stationary temperature of at least one said diagnostic area in such a way as to initiate a reverse transcription of RNA and to amplify a nucleotide sequence selected beforehand, and isothermally;

examining said diagnostic areas to determine the presence or absence of a said target compound.

Advantageously, the third step of the process comprises a nucleotide sequence amplification by RT-RPA (reverse transcription—recombinase polymerase amplification).

Another subject of the invention is a pair of primers which is selected from the group consisting of SEQ ID NOS.: 1-2, SEQ ID NOS.: 4-5; and SEQ ID NOS.: 7-8.

Another subject of the invention is a nucleotide probe selected from the group consisting of SEQ ID NOS.: 3, 6, 9, 13, 14 and 15.

Another subject of the invention is a composition of oligonucleotides comprising a pair of primers and a probe, selected from the group consisting of a pair of primers of sequence SEQ ID NOS.: 1-2 and a nucleotide probe of sequence SEQ ID NO. 3; a pair of primers of sequence SEQ ID NOS.: 4-5 and a nucleotide probe of sequence SEQ ID NO. 6; and a pair of primers of sequence SEQ ID NOS.: 7-8 and a nucleotide probe of sequence SEQ ID NO.: 9.

Another subject of the invention is a process for producing a device, comprising at least the steps consisting in:

forming areas and channels in a said porous substrate by heating said porous substrate for at least one minute at a temperature greater than or equal to 100° C. and preferentially greater than or equal to 150° C.;

depositing, on an area of said porous substrate, at least one element chosen from reactive compounds and target compounds diluted in an aqueous solution conditioned at a temperature of between 0° C. and 10° C., and lyophilizing them

sealing said porous substrate in a plastic sheet.

The invention will be understood more clearly and other advantages, details and features thereof will emerge during the explanatory description which follows, and which is given by way of example with reference to the appended drawings in which:

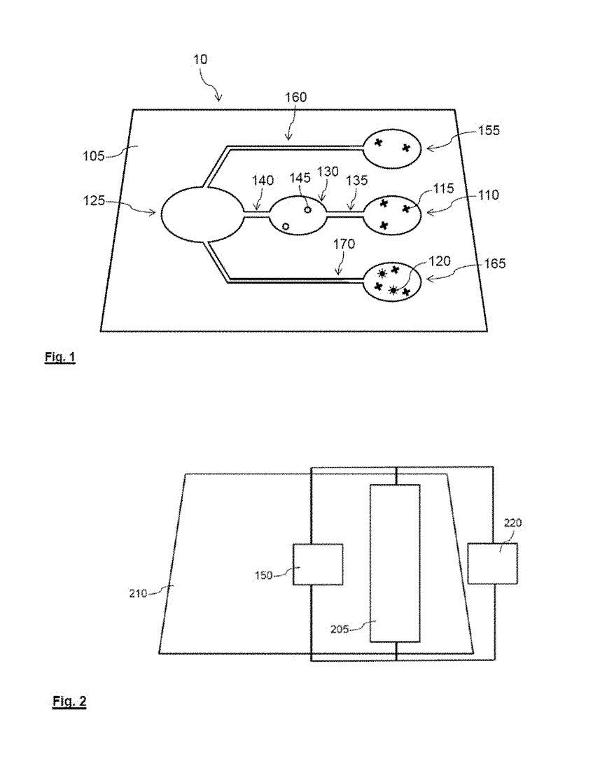

FIG. 1 diagrammatically illustrates, in perspective, one embodiment of the device according to the invention;

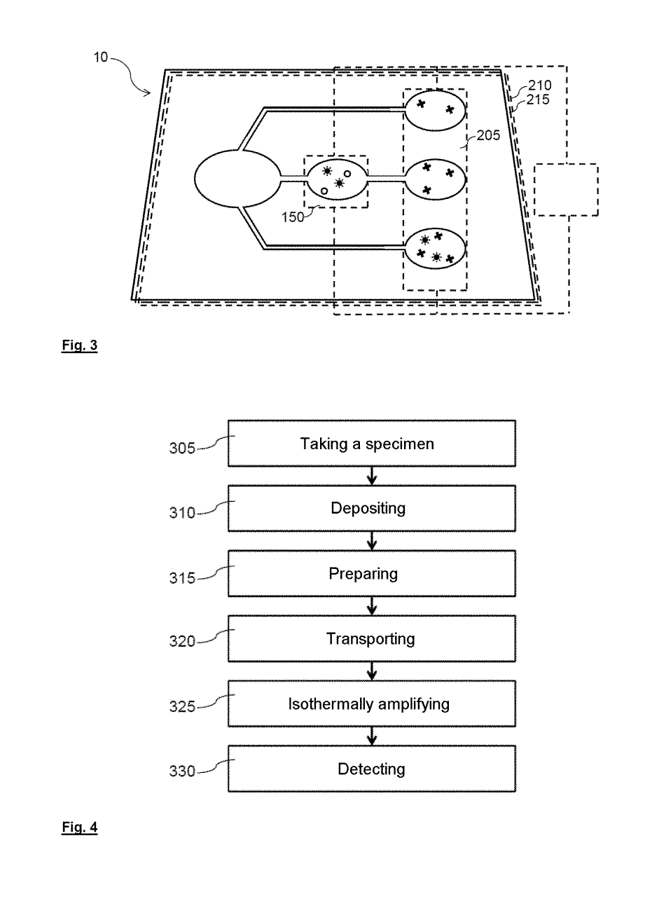

FIG. 2 illustrates one particular embodiment of the means for locally conditioning temperature and of the support layer according to the invention;

FIG. 3 illustrates a device according to the invention assembled for carrying out a diagnosis;

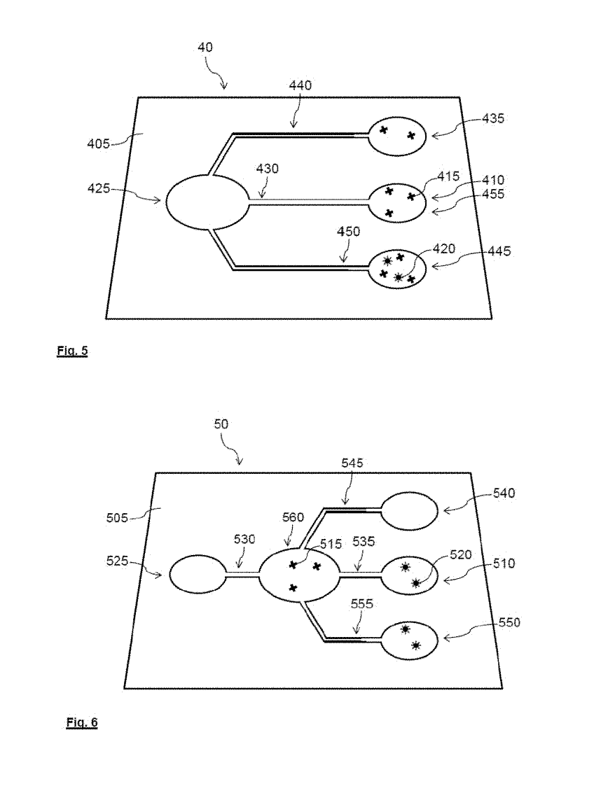

FIG. 4 illustrates a flow diagram of steps of the process which is the subject of the invention;

FIG. 5 illustrates one embodiment of the device according to the invention;

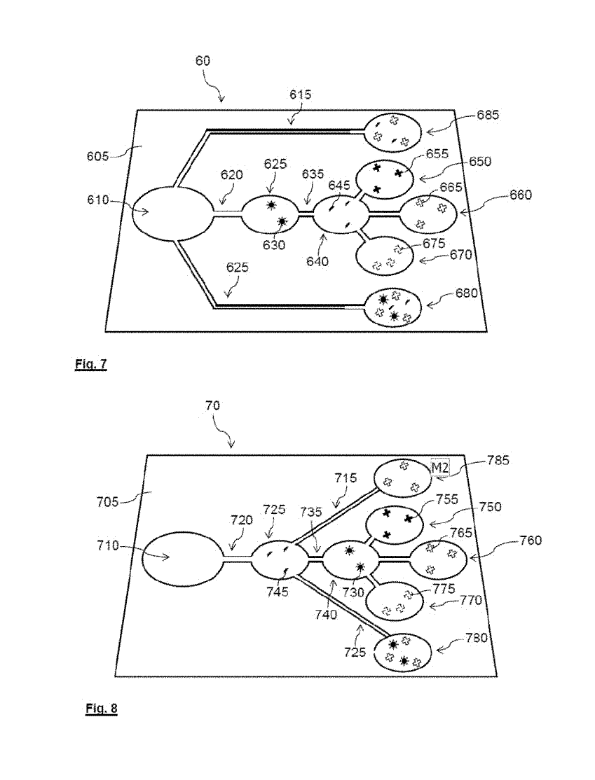

FIG. 6 illustrates one particular embodiment of the device according to the invention;

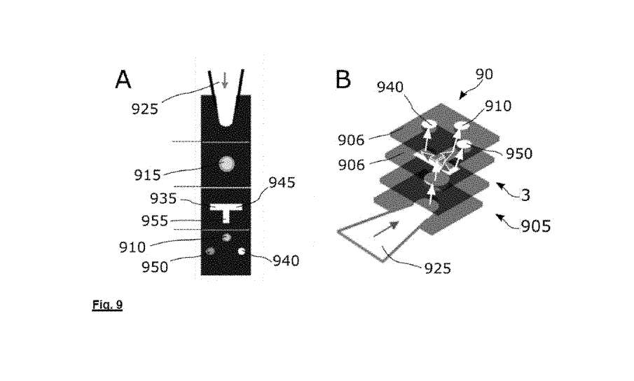

FIG. 7 illustrates one particular embodiment of the device 60 according to the invention;

FIG. 8 illustrates one particular embodiment of the device 50 according to the invention;

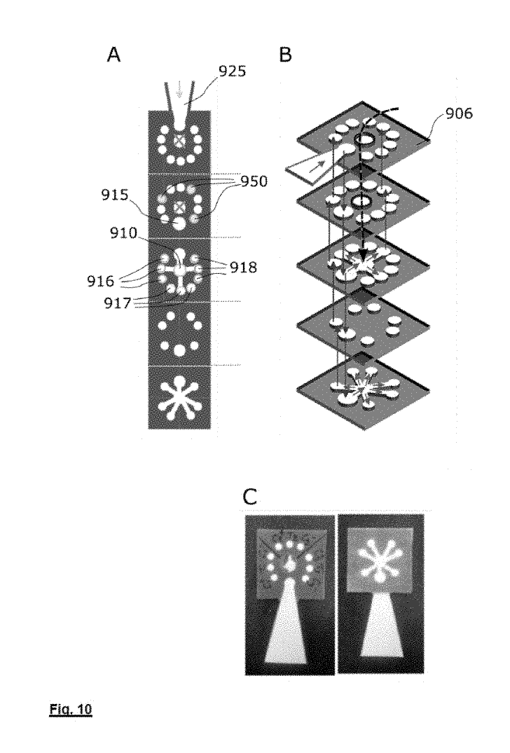

FIG. 9 illustrates several embodiments of the device according to the invention;

FIG. 10 illustrates one embodiment of the device according to the invention;

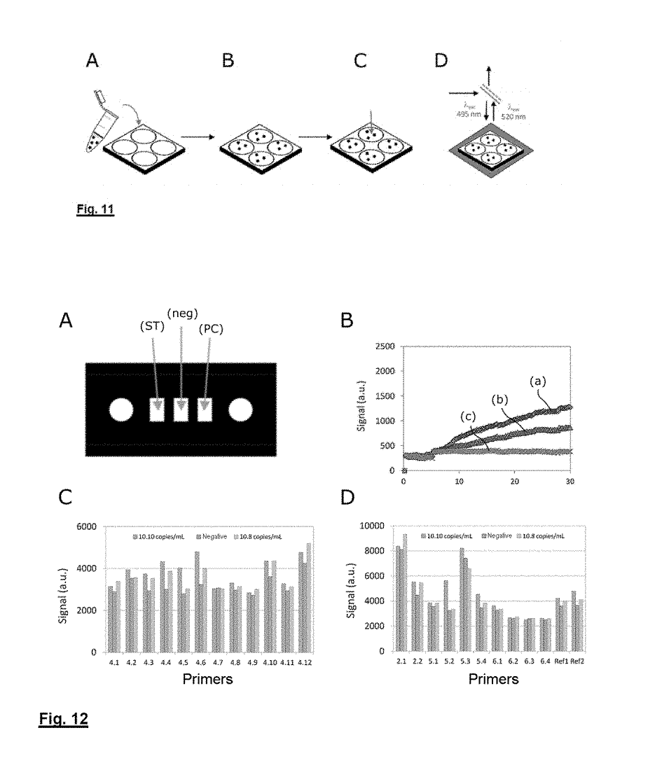

FIG. 11 illustrates a general process for amplifying DNA on a paper substrate;

FIG. 12 illustrates a paper device suitable for carrying out an amplification reaction of RT-RPA type according to one embodiment of the invention;

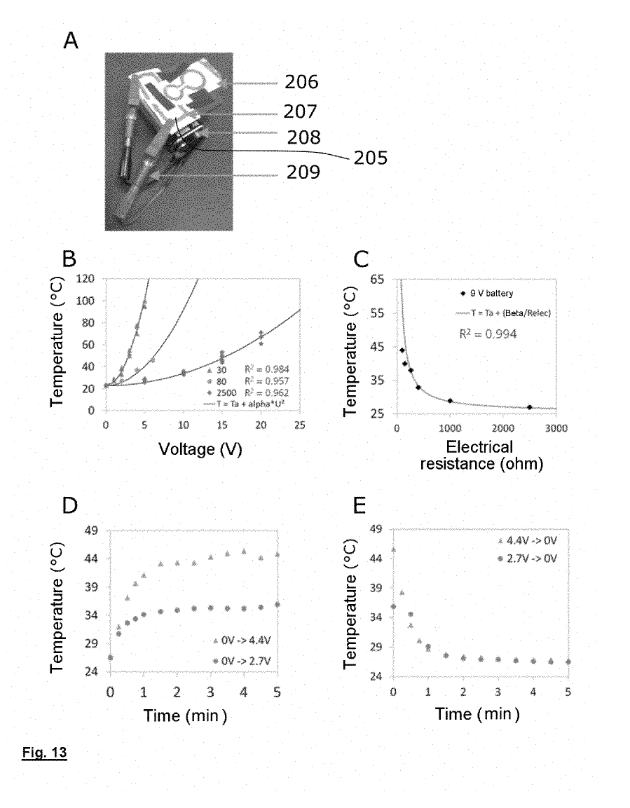

FIG. 13 illustrates a different paper device according to the invention and a means for locally conditioning temperature according to one embodiment of the invention;

FIG. 14 illustrates the dependence of the material of the areas or channels in contact with the reactive compounds of the RT-RPA on the result of an amplification reaction;

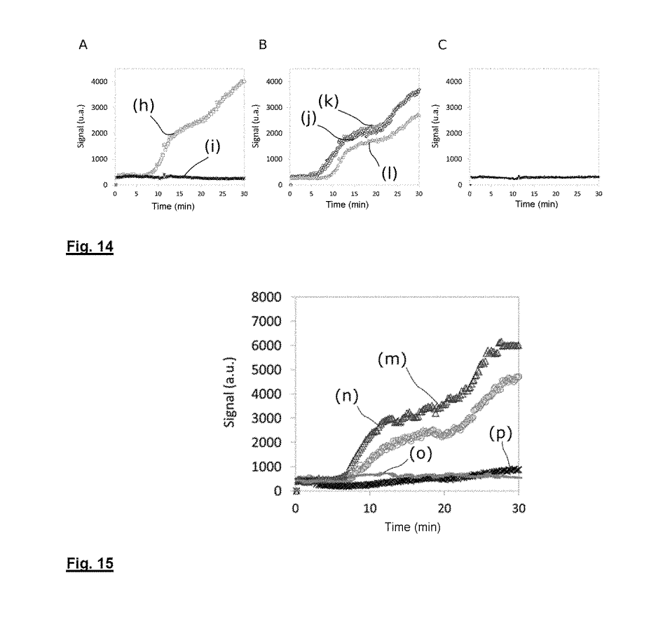

FIG. 15 illustrates the influence of a paper substrate on the kinetics of fluorescence signal emitted during an RT-RPA;

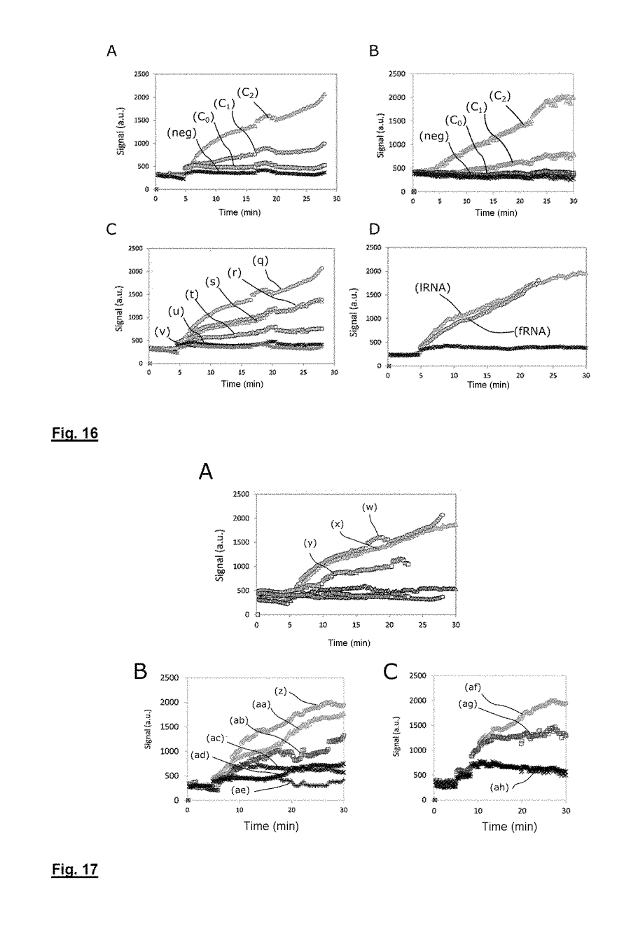

FIG. 16 illustrates the influence of wax or PDMS barriers, of the serum and of the lyophilization of the target RNA compound on the kinetics of fluorescence signal emitted during an RT-RPA on paper;

FIG. 17 illustrates the storage of a device for amplification by RT-RPA on paper;

FIG. 18 illustrates the dependence between the amount of reactive compounds deposited on the paper substrate and the geometry of the substrates in question;

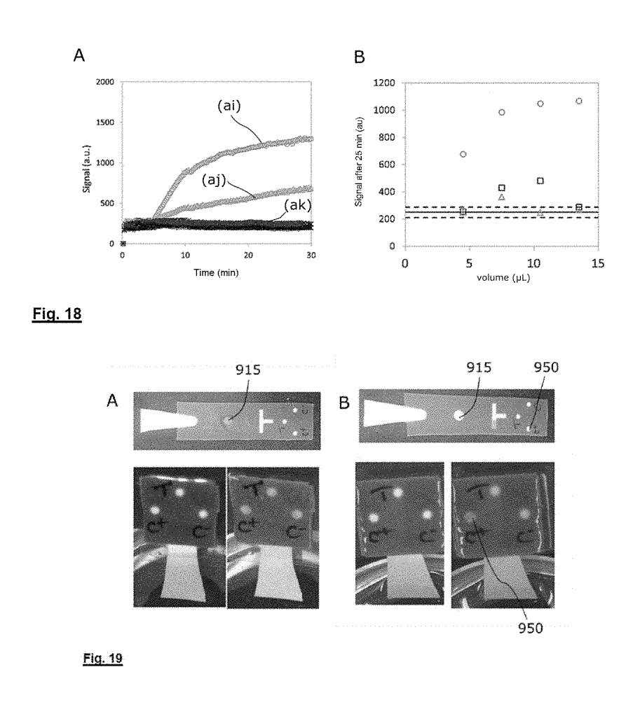

FIG. 19 illustrates the flows in a device according to the invention;

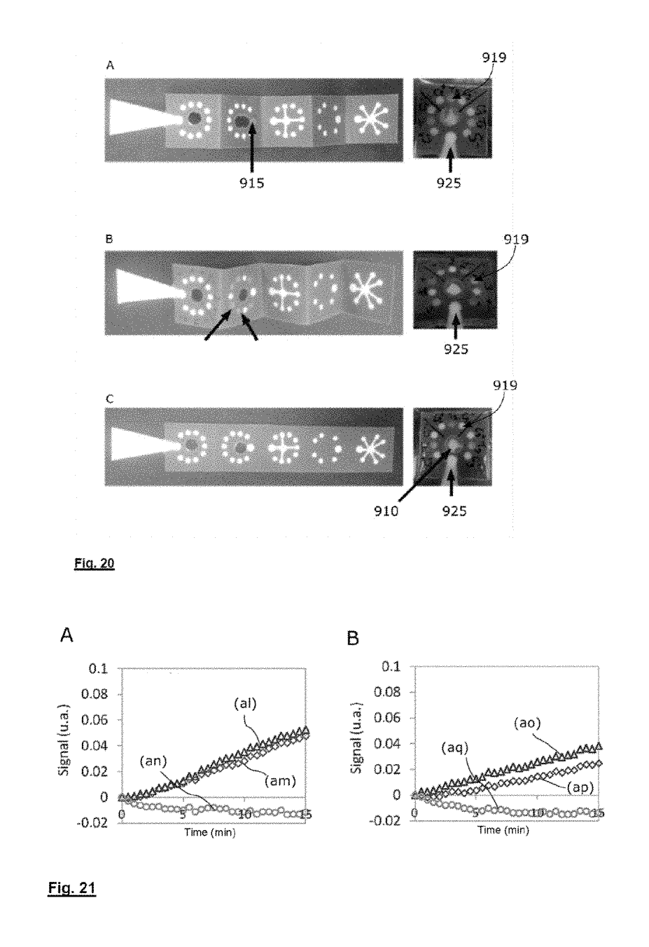

FIG. 20 illustrates the flows in a device according to the invention;

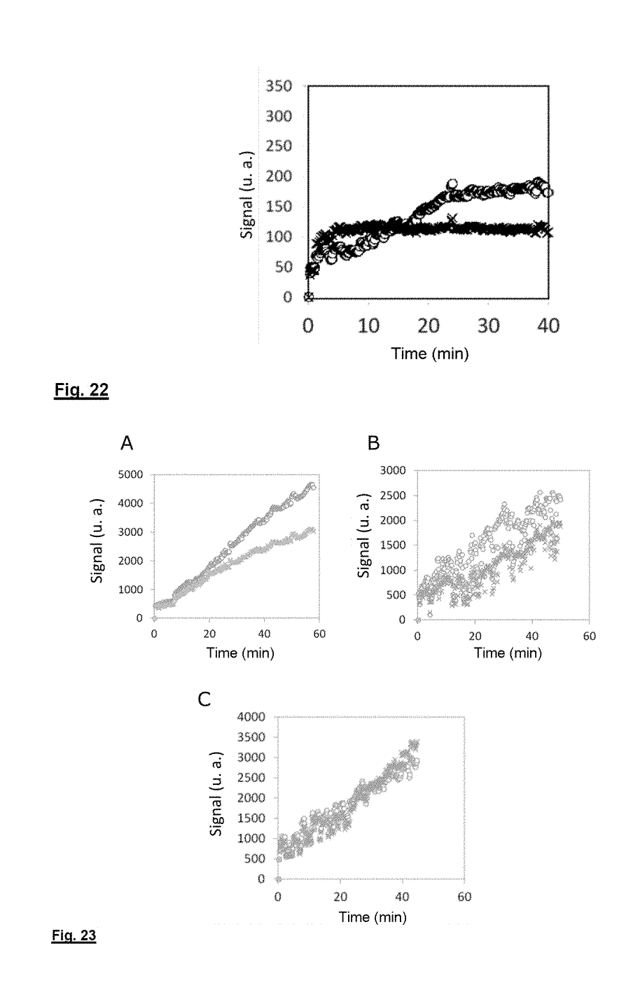

FIG. 21 illustrates a detection of the RNA of an Ebola virus;

FIG. 22 illustrates the dependence of the conditioning of the reactive compounds and of the target compounds 920 on the kinetics of fluorescence emission of an RT-RPA;

FIG. 23 illustrates the dependence of the conditioning of the reactive compounds and of the target compounds 920 on the kinetics of fluorescence emission of an RT-RPA;

FIG. 24 illustrates tests resulting in the choice of the primers;

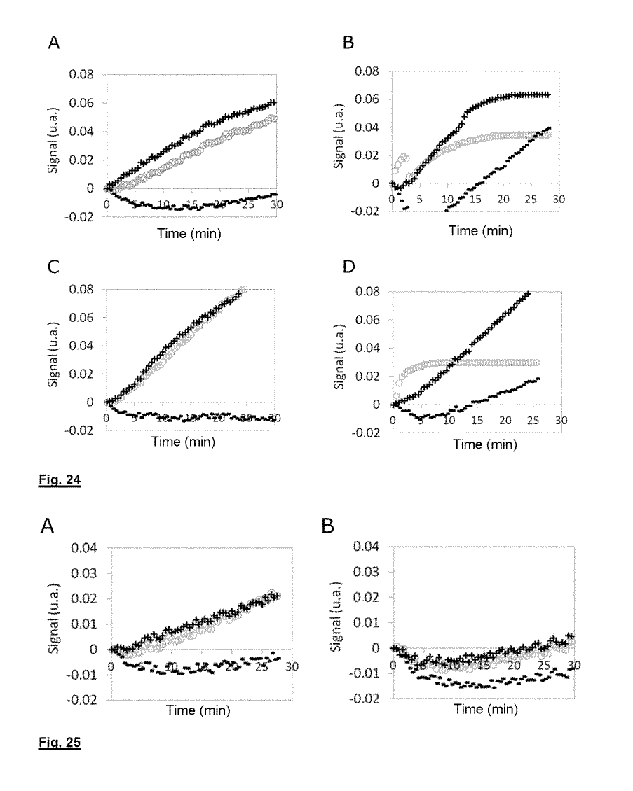

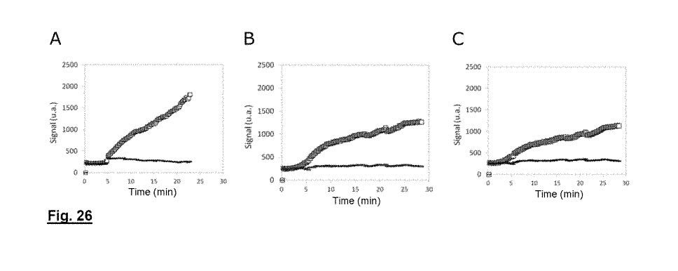

FIG. 25 illustrates tests resulting in the choice of the primers;

FIG. 26 illustrates the effect of the lyophilization of target compounds 920 and of an RNAse inhibitor on an RT-RPA reaction on paper.

The following description presents several implementation examples of the device of the invention: these examples do not limit the scope of the invention. These implementation examples present both the essential features of the invention and also additional features associated with the embodiments in question. In the interest of clarity, the same elements will bear the same references in the various figures.

FIG. 1 diagrammatically illustrates, in perspective, one embodiment of the device 10 which is a subject of the present invention. This device 10 for diagnosis by DNA amplification comprises:

a porous substrate 105 comprising:
an area 110 comprising at least one reactive compound 115 which reacts with a target compound 120 of a sample, referred to as the “diagnostic area”;
an area 125 for depositing a carrier fluid transported, by capillarity, into an area 130 for depositing a sample and into the diagnostic area 110;
the area 130 for depositing a sample, positioned between the area 125 for depositing a carrier fluid and the diagnostic area 110, which comprises a compound 145 for preparing the sample by chemical lysis of the cells;
two channels, 135 and 140, positioned in the thickness of the porous substrate, respectively between, on the one hand, the area 125 for depositing a carrier fluid and the area 130 for depositing a sample, and, on the other hand, the area 130 for depositing a sample and the diagnostic area 110;
an additional area 155 comprising at least one reactive compound 115 of the diagnostic area 110 linked by a channel 160 to the area 125 for depositing a carrier fluid, and
an additional area 165 comprising at least one reactive compound 115 of the diagnostic area 110, and at least one target compound 120, linked by a channel 170 to the area 125 for depositing a carrier fluid;

a means 205 for locally conditioning the temperature of the diagnostic area 110 and of the additional control area 155 and additional control area 165;

a means 150 for locally conditioning the temperature of the area 130 for depositing a sample;

a support layer 210 for the conducting electrical circuit; and

a leaktight layer 215, the leaktight layer 215 being positioned so as to be inserted between the porous substrate 105 and the support layer 210.

In particular, in FIG. 1, the porous substrate 105 used by the device 10 can be observed. This porous substrate 105 is, for example, a sheet made of paper, of cellulose fibers, of nitrocellulose membranes or of filter paper constituting a porous medium capable of allowing the passage of a carrier fluid. On contact with the porous substrate 105, a drop (for example of water, of blood or of buffer solution) diffuses at the surface and in the thickness of the porous substrate 105 around the point of contact between the drop and the porous substrate 105. In order to channel the flow of the drops in this porous substrate 105, a set of areas and of channels is defined in the porous substrate 105. These areas and channels are delimited by barriers passing through the porous substrate 105 in its thickness. These barriers are formed, for example:

by deposition of molten wax penetrating into the thickness of the porous substrate and then solidified (the solid wax impregnating parts of a porous substrate 105 can delimit one or more channels);

by a hydrophilic-hydrophobic contrast in the thickness of the porous substrate 105;

by laser cutting;

by crosslinking of polymers and/or

by photolithography in the thickness of the porous substrate 105.

The term “area” refers to a closed volume of the porous substrate 105 comprising at least one opening and the term “channel” refers to a conduit linking at least two area openings.

The device 10 uses several areas in the porous substrate 105: one area, referred to as the “diagnostic area 110”, comprises at least one reactive compound 115 which reacts with the target compound 120. For example, a mixture of reactive compounds 115 is made up of an enzyme, a mixture of the four deoxyribonucleotides and a selection of primers which make it possible to carry out a DNA amplification technique, among:
PCR (“Polymerase chain reaction”), RT-PCR (“reverse transcriptase—PCR) and derivatives thereof;
LAMP (“Loop-mediated isothermal amplification”) and RT-LAMP (“reverse transcription—LAMP”);
RPA (“Recombinase polymerase amplification”) and RT-RPA (“reverse transcription—RPA”);
SDA (“Strand displacement amplification”) and RT-SDA (“reverse transcription—SDA”);
HAD (“Helicase dependent amplification”) and RT-HDA (“reverse transcription—HDA”) and
NEAR (“Nicking enzyme amplification reaction”) and RT-NEAR (“reverse transcription—NEAR”).

An implementation example of RPA is described by Piepenburg et al., “DNA detection using recombination proteins”, PLoS biology, 4(7), 2006. In other embodiments of the invention, the reactive compounds 115 used make it possible to carry out at least one amplification technique described in Yan, L. et al., “Isothermal amplified detection of DNA and RNA”, Molecular BioSystems, 10(5), 970-1003, 2014.

At least one reactive compound is present in lyophilized form in the diagnostic area 110: it can be arranged at the surface of the porous substrate 105, and/or in the pores of the porous substrate 105 (that is to say in the porous substrate 105). In variants, at least one reactive compound is present in dried form and/or in the form of a hydrogel. In other variants, at least one reactive compound is present in wet format. In these variants, the diagnostic area 110 is isolated from the external environment by plastic sheets, for example.

When at least one reactive compound is in lyophilized form, each said reactive compound is rehydrated by the carrier fluid deposited in the area 125 for depositing a carrier fluid.

When at least one reactive compound comes into contact with the target compound 120, with the proviso of appropriate thermal conditions, the primers hybridize to the DNA strand which is complementary thereto, and the enzyme can then bind to the primers and replicate the DNA strand (the available deoxyribonucleotides are used to create the strands complementary to the DNA strand). During this replication, a colorimetric or fluorescent intercalating agent is generally integrated and is responsible for the detection signal.

In the case of a colorimetric detection, at least one reactive compound 115 changes color on contact with the target compound 120, which makes it possible to detect the presence of the target compound. If a target compound is detected, a user of the device 10 can deduce therefrom the presence or absence of a microorganism and deduce the infection of an individual by said microorganism of which the DNA is the target compound 120 of the reactive compounds 115 of the device 10.

In variants, at least one reactive compound 115 emits a fluorescence signal on contact with the target compound 120.

This diagnostic area 110 is linked to the area 130 for depositing a sample by a channel 135 which is preferably rectilinear. The area 130 for depositing a sample can comprise a compound 145 for preparing the sample by lysis of the cells, which is present in dry or lyophilized form in the thickness of the porous substrate 105. In the case of a chemical lysis, this compound 145 is, for example, a detergent agent which destroys the plasma membrane of the cells present in the sample, thereby making it possible to extract the generic material from the cells.

The area 130 for depositing a sample is linked to the area 125 for depositing a carrier fluid by a channel 140. The area 125 for depositing a carrier fluid is configured to receive a carrier fluid, for example. This area 125 for depositing a carrier fluid is linked by a channel 160, different than the channel 140, to an additional area 155 comprising at least one reactive compound 115 (for example having the same composition as the reactive compounds 115 of the diagnostic area 110). This area 155 termed “additional verification area 155” has the function of verifying that the carrier fluid deposited in the area 125 for depositing a carrier fluid does not contain the target compound 120, which would induce an erroneous diagnosis with respect to the sample deposited in the area 130 for depositing a sample. In other configurations of the channels and of the areas of a device according to one embodiment of the invention, a verification of the absence of a target compound in the carrier fluid can be carried out in a device in which an area 125 and an area 155 are indirectly linked: these two areas can for example be linked via combinations of area(s) and/or of channel(s), in such a way that the flow of carrier fluid 125 does not pass in transit through an area 130, 410, 910 for depositing the sample so as to join an additional area 155. This combination can be a succession of at least one element chosen from a channel and an area other than an area 130, 410, 910 for depositing a sample.

The area 125 for depositing a carrier fluid is also linked, by a channel 170 other than the channel 140 and than the channel 160, to an additional area 165 comprising the reactive compound 115 and at least one target compound 120 in lyophilized form. When the carrier fluid hydrates the additional area 165, the reactive compound 115 reacts with the target compound 120. In general, in devices according to embodiments of the invention, an additional area 165, comprising a reactive compound 115 and a target compound, can be linked to the area 125 for depositing a carrier fluid indirectly, for example by a succession of at least one element chosen from a channel and an area other than an area 130, 410, 910 for depositing a sample. This area 165 referred to as “additional verification area 165” has the function of verifying the operation of the device 10.

In variants, the area 125 for depositing a carrier fluid has an area substantially greater than the areas of the other areas of the device 10. Thus, the depositing of a sample in the area 130 for depositing a sample and then the depositing of a carrier fluid in the area 125 for depositing a carrier fluid leads, by a capillary pump mechanism, to a displacement of the water in the additional verification area 155 and additional verification area 165, and in the area 130 for depositing a sample. The carrier fluid thus transports the sample into the area 130 for depositing a sample and into the diagnostic area 110. The areas and channels of the porous substrate 105 act as a microfluidic system.

The porous substrate 105 is, for example, wrapped in a plastic sheet covering all of the surfaces of the porous substrate 105 with the exception of the depositing areas 125 and 130. In this way, the porous substrate 105 is protected and each reactive compound 115 is isolated in order to prevent contaminations.

FIG. 2 illustrates one particular embodiment of the means 205 for locally conditioning the temperature and the support layer 210. The support layer 210 is, for example, a sheet of paper or of cellulose fibers similar to the material used to produce the porous substrate 105. This support layer 210 has dimensions that are preferentially identical to those of the porous substrate 105.

A thermal conditioning means 205 can comprise a conducting electrical circuit 205 positioned on each support layer 210 in such a way that, when a current passes through the circuit, this circuit locally heats the porous substrate 105 by the Joule effect. The positioning of the conducting electrical circuit 205 is done in such a way that, when the support layer 210 and the porous substrate 105 are brought into contact, the means 205 for locally conditioning the temperature is placed facing the diagnostic areas 110 and the additional verification area 155 and additional verification area 165. The means 205 for locally conditioning the temperature is configured to heat the diagnostic area 110 and the additional control area 155 and additional control area 165 to a temperature of between 20° C. and 120° C. Preferentially, this temperature is between 35° C. and 70° C. and preferentially between 45° C. and 65° C. Other thermal conditioning means can be used, as a variant or in addition, for instance a Peltier-effect device.

A secondary conductor electrical circuit 150 is positioned in such a way as to be facing the area 130 for depositing a sample of the porous substrate 105 in order to act as a means 150 for locally conditioning the temperature for preparing the sample by thermal denaturation of the sample. In general, a conducting electrical circuit 150 can be positioned in such a way as to be facing the other areas of the device 10, 40, 50, 90.

These electrical circuits, 205 and 150, are supplied with power by a battery 220 for example. This battery 220 has, for example, a voltage of 9 volts, which allows the substrate system 105, layer 205 and battery 220 to be portable. In variants, the means 205 for locally conditioning the temperature is configured to carry out a set of thermal cycles in order to carry out a PCR reaction. In these variants, at least one reactive compound 115 is suitable for carrying out this reaction. In variants, an electrical circuit 150 can consist of an electrically conducting ink drawn, deposited or printed on a sheet of paper. This sheet of paper is, in this case, a sheet distinct from the porous substrate 105.

In FIG. 3, the device 10 assembled to carry out a diagnostic can be seen. In this embodiment, a leaktight layer 215 is positioned so as to be inserted between the porous substrate 105 and the support layer 210. The means 205 for locally conditioning the temperature is positioned under the diagnostic areas 110 and the additional areas 155 and 165, while the means 150 for locally conditioning the temperature is positioned under the area 130 for depositing a sample.

In this way, the sample transported into the area 130 for depositing a sample and into the diagnostic area 110 is heated, which allows the enzymes to operate and allows the function of isothermal amplification of DNA to take place. Furthermore, the local isothermal heating of the additional area 165 makes it possible to verify the operation of each reactive compound 115.

The thermal preparation means 150 makes it possible to purify and inactivate the sample deposited in the area 130 for depositing a sample.

The device 10, illustrated for example in FIGS. 1 to 3, can be produced in a controlled, standardized manner in a laboratory or by industrial processes. After production, the device 10 is stored in a hermetic plastic bag and can be stored away from heat, light and moisture so as to allow preservation of the devices 10.

FIG. 4 illustrates a flow diagram of steps of the process which is the subject of the present invention. This process comprises:

a step of taking a specimen 305 of a sample;

a step of depositing 310 of the sample in an area for depositing a sample of the device 10 as described from the viewpoint of FIGS. 1 to 3;

a step of preparing 315 the sample;

a step of transporting 320 the sample prepared;

a step of isothermally amplifying 325 the sample and a step of detecting 330 the presence of a target compound in the sample.

The step of taking a specimen 305 is carried out, for example, with a syringe or with a needle pricking an individual at the end of the finger so as to extract a drop of blood. In variants, a drop of urine, of saliva or of any other biological fluid is taken. In preferential variants, a drop of blood taken from a blood sample is used. In the interests of simplicity of use, a drop of blood taken at the end of the finger of the individual is often used.

The depositing step 310 is carried out, for example, by bringing the sample into contact with the area for depositing a sample of the device as described from the viewpoint of FIGS. 1 to 3.

The preparing step 315 is carried out by lysis of the cells of the sample and/or by thermal denaturation of the sample. The chemical lysis is carried out with a detergent agent, for example, whereas the thermal denaturation is carried out by local isothermal heating of the area for depositing a sample by means of a conducting electrical circuit positioned facing said area. This preparing step 315 has the objectives of extracting the DNA or RNA material from the cells of the sample and of decontaminating the sample by deactivating the microorganism(s) for example. The transporting step 320 is carried out, for example, by depositing a standardized amount of sterile carrier fluid on the area for depositing a carrier fluid of the device 10. This carrier fluid, by means of a capillary pump mechanism, transports the sample into the area for depositing a sample and into the diagnostic area of the device 10.

The volume of carrier fluid deposited is, for example, thirty times greater and preferentially one hundred times greater than the deposited volume of aqueous solution in which the sample is diluted.

The isothermal amplifying step 325 can be carried out by conditioning the temperature of the reactive medium, comprising the sample prepared and each reactive compound, in the diagnostic area. This heating allows the enzymes, initially present in dried and then rehydrated form, to amplify the DNA of the target compound.

The isothermal amplifying 325 is less restrictive than the PCR methods and the thermal cycles associated with these methods.

The means 205 for locally conditioning the temperature is supported by a layer which, combined with the porous substrate of the device 10, forms a chip with three thicknesses:

a layer of porous substrate comprising the microfluidic system of channels and of areas;

an adhesive leaktight layer attached to the porous substrate and

a layer supporting the electrical heating circuit.

The detecting step 330 is carried out, for example, by detecting the color or the fluorescence of the porous substrate at the level of the diagnostic area. This spectrometry is carried out directly by the user or, for example, by a portable communicating terminal, such as a mobile telephone comprising an image sensor for example. Image processing software loaded into said terminal or into a remote computer server determines the color of the porous substrate and deduces diagnostic information therefrom.

In variants, the process comprises a step of verifying the purity of the carrier fluid by detecting the color in an area of the porous substrate in connection with the area of depositing a carrier fluid. If this area, provided only with each reactive compound, has a color or a fluorescence characteristic of the presence of the target compound in said area, the carrier fluid used is infectious and the diagnosis established by the device 10 cannot be exactly accurate.

In variants, the process comprises a step of verifying the operation of the device 10 by detecting the color or the fluorescence in an area of the substrate comprising, in lyophilized forms, each reactive compound and the target compound. When the carrier fluid deposited hydrates this area, the compounds are brought into contact and the color of said area becomes representative of the detection of the target compound. If this area does not turn this color, then the diagnosis carried out cannot be considered to be correct.

Once the diagnosis has been established, the device can be burnt so as to avoid generating infectious waste. The means 205 for locally conditioning the temperature can be extracted so as to be reused in connection with another porous substrate.

FIG. 5 illustrates one particular embodiment of the device 40 which is a subject of the present invention. This device 40 differs from the device 10 as described from the viewpoint of FIG. 1 in that:

the area 410 for depositing a sample and the diagnostic area 455 are combined together and in that

the two channels are combined together in a single channel 430.

Thus, this device 40 comprises a porous substrate 405 similar to the porous substrate 105 described from the viewpoint of FIG. 1, and a set of channels 430, 440 and 450, and of areas 410, 425, 435, 445 and 455, which are delimited on and in this porous substrate 405.

The area 410 corresponds to an area for depositing a sample. This area 410 for depositing a sample is linked to an area 425 for depositing a carrier fluid similar to the area 125 for depositing a carrier fluid described from the viewpoint of FIG. 1. In this embodiment, the carrier fluid serves mainly to hydrate the area 410 for depositing a sample and diagnostic area 455 in order to prevent drying of the compounds before a diagnosis is carried out.

The additional areas, 435 and 445, correspond respectively to the areas 155 and 165 described from the viewpoint of FIG. 1 and provide similar functions. In variants, the area 410 for depositing a sample and diagnostic area 455 comprise a compound for preparing the sample similar to the compound 165 for preparing the sample described from the viewpoint of FIG. 1.

FIG. 6 illustrates one particular embodiment of the device 50 which is a subject of the present invention. This device 50 comprises:

a porous substrate 505;

an area 525 for depositing a carrier fluid linked to a channel 530 for transporting the carrier fluid by capillarity to an area 560 comprising at least one reactive compound 515;

three channels, 535, 545 and 555, for transporting the carrier fluid and each reactive compound 515 by capillarity respectively from the area 560 to:
an area 510 for depositing a sample which acts as a diagnostic area during the contact between the sample and each reactive compound 515 under the action of a means 205 for locally conditioning the temperature (not represented);
an additional area 540 comprising neither reactive compound 515, nor target compound 520, for verifying that the carrier fluid does not comprise any target compound 520, and
an additional area 550 comprising the target compound 520 for verifying the operating capacity of the device 50.

FIG. 7 illustrates one particular embodiment of the device 60 which is a subject of the present invention. In this FIG. 7, three components of the reactive compounds described above are distinguished. These reactive compounds comprise:

primers, that is to say small DNA strands which specifically recognize a DNA or RNA sequence that is sought in a sample, this sequence being given the name “target compound” in the figures above;

deoxyribonucleic bases, that is to say the elementary bricks which form the DNA and

at least one type of enzyme capable of assembling the deoxyribonucleic bases so as to form the DNA strand complementary to the target being sought. The enzyme operates only if the primer has found a complementary sequence.

This device 60 comprises a porous substrate 605 comprising an area 610 for depositing a carrier fluid linked to three channels, 615, 620 and 625, for transporting the carrier fluid by capillarity.

The carrier fluid is transported, by the channel 615, to an area 685 comprising a pair of primers 665, deoxyribonucleic bases and enzymes 645, in lyophilized form for example, for verifying that the carrier fluid does not comprise any target compound 630. If the target compound 630 is present, the primers 665 and enzymes 645 react with the target compound 630 and indicate to a user, by means of a fluorescent probe for example, that the target compound 630 is present in the carrier fluid.

The carrier fluid is transported, by the channel 625, to an area 680 comprising a pair of primers 665, deoxyribonucleic bases, enzymes 645, and the target compound 630 in lyophilized form, for example. On contact with the carrier fluid, the primers 665, the enzymes 645 and the target compound 630 react, indicating to a user, by means of a fluorescent probe for example, that the device 60 is operating appropriately in the presence of the target compound 630.

The carrier fluid is transported, by the channel 620, to an area 625 for depositing a sample, this sample comprising in this case the target compound 630. This area 625 for depositing a sample is linked, by a channel 635, to an area 640 for storing enzymes 645 in lyophilized form.

The carrier fluid transports the sample and the stored enzymes 645 into three distinct areas, 650, 660 and 670, each linked to the storage area 640 by a distinct channel. The term “linked” elements is intended to mean that there is an arrangement of area(s) and/or of channel(s) capable of transporting a carrier fluid from one element to the other element(s), by capillarity and/or by another mode of fluid flow in/on a porous substrate 105 between said elements. A different pair of primers, 655, 665 and 675, is placed in each area 650, 660 and 670. Each pair of primers, 655, 665 and 675, corresponds to a different diagnosis, each pair of primers, 655, 665 and 675, reacting with a different target compound 630 or with a different part of the target compound 630.

FIG. 8 illustrates one particular embodiment of the device 70 which is a subject of the present invention. In this FIG. 8, three components of the reactive compounds described above are distinguished. These reactive compounds are made up:

of primers, that is to say of small DNA strands, referred to as “oligonucleotides”, which specifically recognize a sequence being sought in a sample, this sequence being called “target compound” in the figures above. These primers can be suitable for detecting the presence of a pathogenic agent, for example by RT-RPA;

of deoxyribonucleotides (dNTPs);

of enzymes capable of assembling the deoxyribonucleotides so as to form the DNA strand complementary to the target compound. These enzymes can be polymerases, the operation of which requires, inter alia, that the primer find a complementary sequence. Other enzymes may be required, in particular during the implementation of an isothermal amplification reaction by RT-RPA in the device 10, 40, 90. During the implementation of an RT-RPA, the enzymes may be single-stranded binding proteins (SSBs) and/or recombinases having the function of hybridizing primers with homologous DNA sequences. Retrotranscriptases are used to detect a pathogen based on RNA, in particular a pathogen with an RNA genome.

This device 70 comprises a porous substrate 705 comprising an area 710 for depositing a carrier fluid linked to a channel 720 for transporting the carrier fluid by capillarity. The carrier fluid is transported, by the channel 720, to an area 725 for storing enzymes 745 in dry form. This storage area 725 is linked to three channels, 715, 725 and 735.

The carrier fluid comprising enzymes 745 is transported, by the channel 715, to an area 785 comprising a pair of primers 765 and deoxyribonucleotides (dNTPs), in lyophilized form for example, for verifying that the carrier fluid does not comprise the target compound 730. If the target compound 730 is present, the primers 765 and enzymes 745 react and indicate to a user, by means of a fluorescence for example, that the target compound 730 is present in the carrier fluid.

The carrier fluid comprising enzymes 745 is transported, by the channel 725, to an area 780 comprising a pair of primers 765, deoxyribonucleotide bases and the target compound 730 in lyophilized form, for example. On contact with the carrier fluid, the primers 765 and enzymes 745 react and cause a fluorescence indicating to a user that the device 70 is operating appropriately in the presence of the target compound 730.

The carrier fluid comprising enzymes 745 is transported, by the channel 735, to an area 740 for depositing a sample, this sample comprising in this case the target compound 730.

The carrier fluid transports the sample and the stored enzymes 745 into three distinct areas, 750, 760 and 770, each linked to the storage area 740 by a distinct channel. A different pair of primers, 755, 765 and 775, is placed in each area 750, 760 and 770. Each pair of primers, 755, 765 and 775, corresponds to a different diagnosis, each pair of primers, 755, 765 and 775, reacting with a different target compound 730 or with a different part of a target compound 730.

FIG. 9 illustrates several embodiments of the device according to the invention. Panel A of FIG. 9 illustrates a geometry viewed from above of units used to produce a device according to the invention. In this implementation example, the porous substrate comprises a stack 3 of four primary porous substrates 906, each said primary porous substrate 906 comprising at least one element chosen from a channel and an area. More generally, the porous substrate comprises a stack 3 of at least two primary porous substrates 906, each primary porous substrate 906 comprising a channel and/or a part of a channel and/or an area. This implementation example is suitable for carrying out the test of a sample comprising a negative control, that is to say a control for verifying that the carrier fluid does not comprise any target compound 920, and a positive control, that is to say a control for verifying the operation of the device 90 and of the DNA amplification reaction.

The porous substrate 905 used in this implementation example is produced from grade-1 Whatman (registered trademark) paper for chromatography. Wax units (108R0090/108R0091/108R0092/108R0093, Xerox, registered trademark) are printed (Xerox 8570 ColorQube printer) on the paper and then, for example, heated for one minute at 150° C. on a hotplate (Ika, registered trademark, RCT basic). The wax can thus penetrate the pores of the porous substrate 905. This production step advantageously allows the inactivation of enzymes such as RNAses possibly present in the paper.

In this implementation example of the invention, the device is produced by an alignment of the units followed by folding of the paper. The folding of the paper makes it possible to obtain the stack 3 of several primary porous substrates 906, the units of which are produced by printing. In general, the stack 3 or a part of the stack 3 of primary porous substrates 906 of a device according to one embodiment of the invention can be obtained by folding an initial porous substrate. Each primary porous substrate 906 can be joined to another with good contact by applying a piece of double-sided adhesive tape (Tesa, registered trademark) around the flow units. Plastic sheets (RT2RR, Sigma, registered trademark) make it possible to close and isolate the device. Holes are made using a stamp with a diameter of 5 mm in the plastic coating so as to allow access to the inlets and to the outlets of the device 90, for example for depositing a carrier fluid. It is possible to produce a reservoir for depositing a carrier fluid by coating the stack 3 with several layers of plastic film and then piercing them at an inlet or an outlet. Subsequently, the reservoir(s) produced can be covered with a layer of plastic film, for example to prevent evaporation of a carrier fluid deposited on the device.

In other embodiments of the invention, it is possible to use materials other than wax to produce the barriers of the channels or of the areas. It is for example possible to use known biocompatible materials, such as PDMS (polydimethylsiloxane) for example. The production of PDMS barriers is not as simple as printing wax and can result in a smaller spatial resolution. It will sometimes be used in the following examples implemented for verifying the compatibility of the various materials with the biological reactions carried out in a device.

The dashed lines of panel A of FIG. 9 indicate the directions of the folding during the use of the device as previously described. The four primary porous substrates 906 are aligned during the folding so as to allow vertical openings linked to the areas defined by the units. The components of the various fluid flows in the device can thus be directed in the main plane of a primary porous substrate 906 and also in the plane normal to the main plane of a primary porous substrate 906.

Advantageously, the geometry of the area for depositing a carrier fluid is suitable for being dipped into said carrier fluid.

The device 90 comprises:

an area 925 for depositing a carrier fluid, suitable for being in contact, after stacking 3 of the primary substrate of said area 925, with

an area 915 comprising at least one reactive compound;

three channels 935, 945 and 955 suitable for transporting a carrier fluid and reactive compounds from the area 915 to:
an area 910 for depositing a sample;
an additional area 940 comprising no compound before carrying out a test, making it possible to verify that the carrier fluid does not comprise any target compound 920 (area referred to as negative control);
an additional area 950 comprising target compounds 920 (for example a short RNA fragment comprising a sequence of Ebola virus RNA) suitable for verifying the operation of the device 90 and of the DNA amplification reaction (area referred to as positive control).

Panel B of FIG. 9 illustrates schematically the arrangement of the various primary porous substrates 906 of a device according to the invention, after folding of the unit described in panel A of FIG. 9. The various primary porous substrates 906 are separated in the diagram in order to understand the flows, differently than their arrangement in the device. The gray arrows illustrate the flow in a primary porous substrate 906 and the white arrows illustrate the flow from one primary porous substrate to another. According to this embodiment of the invention, the two areas 910 and 940 are in contact by means of the stack 3 of the primary porous substrates 906. A carrier fluid brought into contact with the area 925 for depositing a carrier fluid follows a flow through all of the primary porous substrates 906 from bottom to top. The flow of a carrier fluid in the device can distribute the reactive compounds present in the area 915 in the three channels 935, 945 and 955 and then in the area 910 for depositing a sample, in the additional negative control area 940 and in the additional positive control area 950 comprising target compounds 920. In general, each of the areas of the device according to the invention is linked to the other areas by at least one element chosen from a channel and an area: this feature allows a carrier fluid deposited on an area 125, 525, 925 for depositing a carrier fluid to be transported into each of the areas of the device from a single localized deposit.

Advantageously, the presence of several test areas (according to this embodiment, areas 910, 940 and 950) on the same device makes it possible to carry out several experiments in parallel under identical temperature, hygrometry, etc., conditions and thus to perform a significant comparison of the detection results.

Advantageously, at least one said compound (for example a reactive compound and/or a target compound 920) is present in at least one of said areas in lyophilized form. The lyophilization of the various compounds can be carried out for example for 2 hours at −80° C. in a lyophilizer. After depositing of the reactive compounds and lyophilization thereof, the stack 3 can be prepared by folding, and the device can be covered with an impermeable sheet, for example made of plastic. The device thus packaged can be stored at ambient temperature, away from moisture and light, for several days or several weeks. During the test, the experiment consists in depositing the sample in the area 910 for depositing a sample, closing the openings with an impermeable sheet and immersing the area for depositing a carrier fluid in said carrier fluid (that is to say, more generally, bringing a carrier fluid into contact with the area 125, 425, 925 for depositing a carrier fluid), for example a buffer solution. After carrying out the biological reactions, the experimenter or the user can examine diagnostic areas 110, 410 to determine the presence or absence of a said target compound 120, 420, 920 in the sample. In some cases, direct visualization by the user may be impossible because of the configuration of the device. The visualization of the result can then be indirect, by visualizing colored or fluorescent labels on other areas of the device, coming from a diagnostic area.

In this implementation of the invention, a carrier fluid comprising labels has been used to visualize the flow in the device. This method makes it possible to verify the flow of the reactive compounds to the test areas, the absence of mixing between the flow lines of the test of the sample, and of the positive and negative controls and also the paths of the various compounds. Fluorescein (RAL Reactants, registered trademark), Brillant Blue G (Sigma Aldrich) and Allura Red AC (Sigma Aldrich) are used as labels at a concentration by weight of 10⁻³ g·ml⁻¹. For each label, a drop of 5 μl is deposited on the surface to be labeled, then dried. A carrier fluid, such as water, causes desorption of the labels and allows them to be transported.

FIG. 10 illustrates one embodiment of the device according to the invention. Panel A of FIG. 10 illustrates a geometry viewed from above of units used to produce a device according to the invention. In this implementation example, the porous substrate comprises a stack 3 of five primary porous substrates 906, each said primary porous substrate 906 comprising at least one element chosen from a channel and an area. This implementation example is suitable for carrying out multiplex tests of a sample, each of the tests comprising a negative control and a positive control. The dashed lines of panel A of FIG. 9 indicate the directions of the folding during the production of the device. The crosses indicate areas that have been pierced (absence of substrate) before the folding of the primary porous substrate 906.

In this embodiment, the device comprises:

an area 925 for depositing a carrier fluid;

an area 915 comprising at least one reactive compound;

nine areas comprising:
three areas 916 comprising primers specific for the detection of a first DNA sequence,
three areas 917 comprising primers specific for the detection of a second DNA sequence, and
three areas 918 comprising primers specific to the detection of a third DNA sequence.

A pair of primers can, for example, be suitable for detecting the Ebola virus;

an area 910 for depositing a sample;

three additional areas 950 comprising at least one target compound 920 (for example a short RNA fragment comprising a sequence of the RNA of an Ebola virus in this implementation) suitable for verifying the operation of the device 90 (area referred to as positive control).

Panel B of FIG. 10 illustrates schematically the arrangement of the various primary porous substrates 906 of the device according to the invention, after folding of the primary porous substrate 906 described in panel A of FIG. 10. The various primary porous substrates 906 are separated in the diagram in order to understand the flows, differently than their arrangement in the device. The solid arrows represent diagrammatically various flows during the use of the device. The dashed arrow indicates the area where the sample is deposited during the test.

In this embodiment, a carrier fluid is pumped by the area 925 for depositing a carrier fluid. It conveys and distributes the reactive compounds present in the area 915 into a control line and into a sample line. The term “a line” is intended to mean a series of at least one channel and/or of one area. These two lines distribute the flow to three test areas, three positive control areas and three negative control areas. The sample line contains an area 910 for depositing the sample and three areas 916, 917 and 918 comprising different primers. In this way, the test of a sample may be multiplexed. Likewise, the negative and positive control lines each comprise three areas 916, 917 and 918.

Panel C of FIG. 10 illustrates two photographs of the device according to embodiments of the invention that are described in panels A and B of FIG. 10. The photograph on the left is a view from above of the device. Various visible areas make it possible to observe, for example by fluorescence, the various sample tests, positive controls and negative controls. The photograph on the right is a view from below of the device. It illustrates a part of the positive and negative control lines.

FIG. 11 illustrates a general process for amplifying DNA on a paper substrate. Panel A illustrates the depositing of reactive compounds on circular areas of the paper substrate. The device can then be lyophilized. Panel B illustrates the storing of the device. Said device can for example be stored at ambient temperature for several weeks. Panel C illustrates the initiation of the test step. A carrier fluid, for example water or a buffer solution, can be deposited in the presence or absence of RNA target compounds 920 (respectively positive or negative control), or in the presence of an unknown sample. Panel D of FIG. 11 illustrates the detection by fluorescence of the amplification reactions on paper. A means for conditioning the temperature, for example a Peltier element, conditions the temperature of the substrate between 20° C. and 120° C., preferentially between 25° C. and 65° C., and most preferentially between 30 and 40° C. The result of a test of the sample, of a positive control or of a negative control can advantageously be detected by fluorescence. A quantitative measurement of the fluorescence of the visible areas (located in a primary substrate 906 at the end of the stack 3) is carried out and described in the results illustrated in the following figures using an external light source for the fluorescence excitation (Leica EL 6000), a macroscope (Leica Z16 APO, magnification 0.57×) and a GFP filter (Leica). The filter comprises an excitation filter centered on a wavelength of 470 nm+/−20 nm, a dichroic mirror centered on a wavelength of 500 nm and a suppression filter centered on a wavelength of 525 nm+/−25 nm. This optical assembly is suitable for the detection of optical probes of FAM type (of absorption wavelength 495 nm and emission wavelength 520 nm). The images are detected by an EM-CCD camera (Hamamatsu C900-13), and recorded at the frequency of one image every ten seconds for 30 minutes and an exposure of 75 ms. Synchronization between the aperture of the shutter and the acquisition by the camera (EG R&D Vision Delays generator) prevents bleaching of the probes by interaction with the sample. The data are standardized by subtracting the first image at each sequence. The signal as a function of time corresponds to the measurement of an average of the signal over the surface area of a test area, for each image.

In other implementations of the invention, it is possible to detect the amplification by colorimetry, by electrical, capacitive measurement or by pH measurement.

FIG. 12 illustrates a paper device suitable for carrying out an amplification reaction of RT-RPA type according to one implementation of the invention. In implementations of the invention, the device comprises the reactive compounds 115 for carrying out an amplification reaction by RT-RPA suitable for testing the sample. An example of amplification of an RNA by RT-RPA is described in the literature in Piepenburg, O., Williams, C. H., Stemple, D. L., & Armes, N. A., DNA detection using recombination proteins, PloS biology, 4(7), 2006.

The RT-RPA amplification technique can be adjusted for the detection of a microorganism in a sample by carrying out a reverse transcription of a region of an RNA specific for said microorganism in order to obtain a cDNA (complementary DNA). A diagnostic area 105, 405 of the device can for example be conditioned at a stationary or constant temperature, so as to initiate a reverse transcription of RNA. The cDNA obtained is then amplified by RPA (isothermally). For example, the detection of the cDNA sequences amplified can be carried out by measuring the fluorescence associated with the hybridization of a fluorescent nucleotide probe specific for the region amplified.

In order to illustrate one embodiment of the invention, the inventors of the present application developed primers and nucleotide probes for specifically detecting an Ebola virus.

For this, the inventors used as a basis the genomic sequences of 145 Zaire Ebola viruses (EBOV) available in the sequence databases (Gire, S. K. et al., Scheiffelin, J. S. (2014). Genomic surveillance elucidates Ebola virus origin and transmission during the 2014 outbreak. Science, 345(6202), 1369-1372). On the basis of the alignment of these sequences, they determined that they were the regions conserved within these sequences. On the basis of the consensus sequences, they then designed six pairs of primers which target conserved regions. For each pair of primers, various fluorescent probes were created. In total, the inventors tested twenty-four pairs composed of one pair of primers and one fluorescent probe. The inventors also tested another primers-probe pair known in the literature for allowing the detection of the Ebola virus by RT-RPA using a small amount of RNA (Euler, M. et al. (2013). Development of a panel of recombinase polymerase amplification assays for detection of biothreat agents. J Clin Microbiol. 2013 April 51(4): 1110-7). Three sets of primers and of probes which are associated therewith are subsequently described in the sequence listing (sets consisting of the primers SEQ ID NO.: 1 and SEQ ID NO.: 2, SEQ ID NO.: 4 and SEQ ID NO.: 5, SEQ ID NO.: 7 and SEQ ID NO.: 8). These three sets correspond to two targeted regions of the RNA of an Ebola virus.

In preferred embodiments of the invention, the primers for amplifying a target region of an Ebola virus, the sequence of which is bordered by positions 8661 and 8820, can comprise:

a pair of primers of sequence SEQ ID NOS: 1 and 2, or

a pair of primers which hybridize specifically to the target region, and of which the position of each primer differs by ±5 nucleotides (in particular by ±4 nucleotides, by ±3 nucleotides, ±2 nucleotides, by ±1 nucleotide or by 0 nucleotide) from the position of the primers of sequence SEQ ID NOS: 1 and 2, or

a pair of primers which specifically amplify the target region, and of which the size of the region amplified by these primers differs from ±10 nucleotides (in particular by ±9 nucleotides, by ±8 nucleotides, by ±7 nucleotides, by ±6 nucleotides, by ±5 nucleotides, by ±4 nucleotides, by ±3 nucleotides, ±2 nucleotides, by ±1 nucleotide or by 0 nucleotide) from the size of the target region, or

a pair of primers which specifically amplify the target region, and of which the sequence of each primer is at least 95% (more particularly at least 96%, at least 97%, at least 98%, at least 99% or 100%) identical to the sequence of each primer of sequence SEQ ID NOS: 1 and 2, or

a pair of primers of sequence SEQ ID NOS: 4 and 5, or

a pair of primers which hybridize specifically to the target region, and of which the position of each primer differs by ±5 nucleotides (in particular by ±4 nucleotides, by ±3 nucleotides, by ±2 nucleotides, by ±1 nucleotide or by 0 nucleotide) from the position of the primers of sequence SEQ ID NOS: 4 and 5, or

a pair of primers which specifically amplify the target region, and of which the size of the region amplified by these primers differs by ±10 nucleotides (in particular by ±9 nucleotides, by ±8 nucleotides, by ±7 nucleotides, by ±6 nucleotides, by ±5 nucleotides, by ±4 nucleotides, by ±3 nucleotides, by ±2 nucleotides, by ±1 nucleotide or 0 nucleotide) from the size of the target region, or

a pair of primers which specifically amplify the target region, and of which the sequence of each primer is at least 95% (more particularly at least 96%, at least 97%, at least 98%, at least 99% or 100%) identical to the sequence of each primer of sequence SEQ ID NOS: 4 and 5.

In particular embodiments of the invention, the primers for amplifying a target region of Ebola virus, the sequence of which is bordered by positions 17158 and 17274, can comprise

a pair of primers of sequence SEQ ID NOS: 7 and 8, or

a pair of primers which hybridize specifically to the target region, and of which the position of each primer differs by ±5 nucleotides (in particular by ±4 nucleotides, by ±3 nucleotides, by ±2 nucleotides, by ±1 nucleotide or by 0 nucleotide) from the position of the primers of sequence SEQ ID NOS: 7 and 8, or

a pair of primers which specifically amplify the target region, and of which the size of the region amplified by these primers differs by ±10 nucleotides (in particular by ±9 nucleotides, by ±8 nucleotides, by ±7 nucleotides, by ±6 nucleotides, by ±5 nucleotides, by ±4 nucleotides, by ±3 nucleotides, by ±2 nucleotides, by ±1 nucleotide or by 0 nucleotide) from the size of the target region, or

a pair of primers which specifically amplify the target region, and of which the sequence of each primer is at least 95% (more particularly at least 96%, at least 97%, at least 98%, at least 99% or 100%) identical to the sequence of each primer of sequence SEQ ID NOS: 7 and 8.

In preferred embodiments of the invention, the primers are selected from the group consisting of SEQ ID NOS: 1-2, 4-5 and 7-8.

In particular modes of the invention, the target regions amplified by the pairs of primers are detected, simultaneously or sequentially, by nucleotide probes.

In preferred embodiments of the invention, a nucleotide probe for detecting a target region of an Ebola virus, the sequence of which is bordered by positions 8661 and 8820, can comprise:
a nucleotide probe of sequence SEQ ID NO.: 13, or
a nucleotide probe of sequence SEQ ID NO.: 3, or
a nucleotide probe of which the sequence is complementary to the sequence SEQ ID NO.: 13, or
a nucleotide probe of sequence SEQ ID NO.: 14, or
a nucleotide probe of sequence SEQ ID NO.: 6, or
a nucleotide probe of which the sequence is complementary to the sequence SEQ ID NO.: 14.

In preferred embodiments of the invention, a nucleotide probe for detecting a target region of an Ebola virus, the sequence of which is bordered by positions 17158 and 17274, can comprise:
a nucleotide probe of sequence SEQ ID NO.: 15, or
a nucleotide probe of sequence SEQ ID NO.: 9, or
a nucleotide probe of which the sequence is complementary to the sequence SEQ ID NO.: 15.

Preferentially, the nucleotide probes are tagged with at least one fluorochrome.

In particular embodiments of the invention, a composition comprising a pair of primers and a nucleotide probe is selected from the group consisting of: a pair of primers of sequence SEQ ID NOS: 1-2 and a nucleotide probe of sequence SEQ ID NO.: 3; a pair of primers of sequence SEQ ID NOS: 1-2 and a nucleotide probe of sequence SEQ ID NO.: 13, a pair of primers of sequence SEQ ID NOS: 4-5 and a nucleotide probe of sequence SEQ ID NO.: 6; a pair of primers of sequence SEQ ID NOS: 4-5 and a nucleotide probe of sequence SEQ ID NO.: 14; a pair of primers of sequence SEQ ID NOS: 7-8 and a nucleotide probe of sequence SEQ ID NO.: 9; a pair of primers of sequence SEQ ID NOS: 7-8 and a nucleotide probe of sequence SEQ ID NO.: 15. According to the invention, the nucleotide probes refer to the sequences described by their SEQ ID NOS and to the strands complementary thereto.

In order to validate the diagnostic device, RNA fragments of an Ebola virus are synthesized in vitro. For this, the inventors synthesized a DNA corresponding to each of the target regions of the genome of an Ebola virus that are amplified by the pairs of primers described above. Each of these DNA sequences was cloned into a pRSET vector downstream of a T7 promoter (GeneArt, Life technologies) so as to make it possible to carry out an in vitro transcription of the cloned sequence (MEGAscript kit, Ambion technologies). Previously known amounts of each of these RNAs are used to determine the sensitivity and/or the threshold of detection of the test.

The RT-RPA amplification is carried out using the reagents of the TwistAmp RT-Exo kit, TwistDX, registered trademark). In one implementation of the RT-RPA on paper, reactive compounds are mixed in aqueous solution, deposited on paper at −20° C. and lyophilized at −20° C. for 1 hour and 30 minutes.

Advantageously, the aqueous solution, comprising a mixture of the various reactive compounds and/or of the target compounds, is conditioned at the temperature of 4° C. before being deposited on the porous substrate before the lyophilization. A subject of the invention is a process comprising a step of forming the channels and the areas in a porous substrate 105, as previously described, followed by a step consisting in depositing, on an area of said porous substrate 105, 405, 905, at least one element chosen from reactive compounds 115, 415 and target compounds 120, 420, 920 diluted in an aqueous solution conditioned at a temperature of between 0° C. and 10° C., and then lyophilizing them. The inventors have discovered that the latter two steps are particularly advantageous for succeeding in storing or preserving biological compounds on a porous substrate, with a view to a reaction suitable for detection of RNA or of DNA. A final step can consist in sealing said porous substrate in a plastic sheet, in order to prevent the entry of any compound capable of degrading lyophilized biological species during the storage of the device.

Advantageously, the reactive compounds comprise RNAse inhibitors.

Advantageously, the reactive compounds comprise cryoprotectors. The use of cryoprotectors can make it possible to preserve a normal activity of the various enzymes of the reacted compound after rehydration.

The inventors also tested the influence of blood serum on the efficiency of the RT-RPA amplification in the device. In the experiments of the subsequent figures, the inventors also describe the use of human sera in various tests, comprising dilutions of RNA target compounds 920. Various sera are heated at 95° C. for 5 minutes and cooled on ice for two minutes. The heating step and the deionized water in which it is diluted have the objective of destroying the viral envelope so as to make it possible to render the genomic material accessible. The inventors also noted that the temperature must at least be 95° C. if the heating time is 5 min. If the temperature is lower than 95° C., then the heating time must be extended to obtain the same result.

The inventors also validated a device and a method for detecting an Ebola virus according to embodiments of the invention by analyzing samples of viral RNA of an Ebola virus originating from the plasma of patients. The samples were collected during the epidemic of 2014 in Macenta, in Guinea, and tested on site. The 120 samples collected were tested in parallel with the device of the present application and with a known RT-PCR method (RealStar® Ebolavirus RT-PCR Kit 1.0, Altona Diagnostics) in order to be able to compare the results obtained with the diagnostic device with those of a reference method.

Panel A of FIG. 12 illustrates a device for testing RT-RPA reactions on paper. This device comprises three rectangular test areas (each of the areas having dimensions of 3 mm by 5 mm) and also two circular openings allowing fixing of the device for detection by fluorescence.

Panel B of FIG. 12 illustrates a fluorescence measurement of a test area during an RT-RPA suitable for the amplification of an RNA target compound 920 of an Ebola virus. The curve (a) corresponds to the amplification of a sample, the curve (b) corresponds to the amplification of an RNA target compound 920 that has been lyophilized beforehand with all of the other reactive compounds (positive control) and the curve (c) corresponds to a negative control (absence of RNA target compound 920). These measurements are carried out using the device described in panel A of FIG. 12.

Panel C and panel D illustrate the fluorescence emission during an RT-RPA, corresponding to a series of primers, of probes and of RNA target compounds 920 of an Ebola virus according to the invention. The amplifications are carried out on a device described in panel A of FIG. 12. Three signals are measured, after 30 minutes, for various sets of four elements consisting of an RNA target compound 920 corresponding to an RNA target region of an Ebola virus, of a first primer, of a second primer and of a probe. During the performing of these RT-RPAs on paper, all of the reactive compounds are lyophilized beforehand on or in the paper. The fluorescence emission of the positive control area and of the test area is greater than that of the negative control area (arranged between the other two areas in the device described in panel A of FIG. 12). The negative control makes it possible to verify that the buffer solution used is not contaminated with an RNA target compound 920 and to verify the absence of leaking between the various areas.

A comparison between five RNA target compounds 920 of an Ebola virus is carried out on a series of devices such as those described in panel A of FIG. 12. For each of the sequences of a target compound 920, several combinations of the primers and of the probes are tested. Dilutions of target compounds 920 at the concentrations of 10¹⁰ copies·ml⁻¹ and of 10⁸ copies·ml⁻¹ are respectively deposited in the test area (ST) and in the positive control area (PC). These tests make it possible to carry out a comparison of several reactive compounds involved in the RT-RPA. The results were compared with the results obtained using primers used in the literature.

The inventors have demonstrated that the RT-RPA primers-probe pairs which work in a tube are not those which work on paper. For example, the primers-probe pairs known in the literature for amplifying small amounts of viral RNA are not suitable for the amplification on paper. The choice of primers and probes is not therefore arbitrary.

Advantageously, the porous substrate is made of nontreated cellulose paper such as Whatman paper. The inventors have demonstrated that acid-treated papers and cotton-based papers may be incompatible with isothermal DNA amplification reactions.

FIG. 13 illustrates a different paper device of the invention and a means 205 for locally conditioning the temperature according to one implementation of the invention. Panel A of FIG. 13 is a photograph of the means 205 for locally conditioning the temperature according to the invention. A different device of the invention 206 is placed in contact with the means 205. Advantageously, the means 205 for locally conditioning the temperature is compact and integrated in the device according to the invention. 207 is a line made of conducting material, in this implementation made of nickel, drawn on paper and connected to a 9-volt battery 208. In this embodiment of the invention, said means 205 for locally conditioning the temperature is a conducting electrical circuit placed facing a porous substrate of the device. The line made of conducting material is brought into electrical contact with connectors 209.

Panel B of FIG. 13 illustrates experimental temperature-conditioning results. The temperature measured on three lines 207 made of conducting material is measured as a function of the voltage applied. Each line made of conducting material is characterized by a different electrical resistance: the triangles correspond to an electrical resistance of 30Ω, the disks correspond to an electrical resistance of 80Ω and the diamonds correspond to an electrical resistance of 2500Ω. The means 205 for locally conditioning the temperature is configured so as to fix the temperature of said depositing area 130 and diagnostic area 110 between 20° C. and 120° C., preferably between 25° C. and 65° C., and most preferentially between 30° C. and 40° C. The experimental measurements are in agreement with the theoretical behaviors (black lines).

Panel C of FIG. 13 illustrates experimental results of temperature conditioning. The temperature of the lines made of conducting material is measured as a function of the electrical resistance of said lines at constant voltage. The measurements are in agreement with the theoretical behavior (black line).

Panel D of FIG. 13 illustrates experimental results of temperature conditioning. The kinetics of the temperature of a line made of conducting material is measured by applying a voltage transition of 0 V to 4.4 V (triangles) or of 0 V to 2.7 V (circles).

Panel E of FIG. 13 illustrates experimental results of temperature conditioning. The kinetics of the temperature of a line made of conducting material is measured by applying a voltage transition of 4.4 V to 0 V (triangles) and of 2.7 V to 0 V (circles).

In one implementation example of the invention, the device 10 is heated to 40° C. This temperature conditioning can be carried out on a Peltier element (MJ Research PTC 200) for 30 minutes. In one embodiment of the invention, a means 205 for locally conditioning the temperature integrated in the device, such as the means previously described, is used. The resistance of the line of conducting material is determined by its geometry (thickness, length, width, etc.) and by the conductivity of nickel. The major characteristics are experimentally verified. For example, Ohm's thermal law can be given by formula (1):

$$T = {T}_{amb} + \frac{{R}_{th}}{{R}_{elec}} {U}^{2} \qquad ( 1 )$$

wherein T is the temperature in Kelvin, T_{amb} is the ambient temperature in Kelvin, Rₜₕ is the thermal resistance in Kelvin·Watt⁻¹ and R_{elec} is the electrical resistance in ohms. For a given line of conducting material, Ohm's thermal law becomes:

(T−T_{amb})∝U² (2)

This relationship is illustrated in panel B of FIG. 13. For a given voltage, the temperature increases with the electrical conductance, as illustrated in panel C of FIG. 13. Experimentally, a change in conditioning from one temperature to another takes place in a time of about one minute as illustrated in panels D and E of FIG. 13. The temperature stability depends on the external conditions.

FIG. 14 illustrates the dependence of the material of the areas or of the channels in contact with the reactive compounds of the RT-RPA on the result of an amplification reaction. Indeed, the influence of the interfaces and of the materials constituting the interfaces of a container can have dramatic consequences on the unfolding of a DNA amplification reaction. Panel A of FIG. 14 illustrates the kinetics of a fluorescence signal emitted during an RT-RPA in tubes for a positive control (h) and negative control (i). Panel B of FIG. 14 illustrates the kinetics of a fluorescence signal emitted during an RT-RPA for a positive control in the presence of paper (k), of plastic (j) and of wax (l). Panel C of FIG. 14 illustrates the kinetics of a fluorescence signal emitted during an RT-RPA for a negative control in the presence of paper, of plastic and of wax.

The influence of various materials of the device according to the invention, such as paper, wax and a sheet of plastic, were tested by adding each of these materials to a tube with the reactive compounds of the RT-RPA. In the presence of an RNA target compound 920, this test makes it possible to detect the inhibitory effects of a material. In the absence of an RNA target compound 920 of an Ebola virus, this test makes it possible to verify that a material is not responsible for an unwanted increase in fluorescence noise. The results presented in FIG. 14 illustrate DNA amplifications by RT-RPA in which an RNA target compound 920 is used at a concentration of 10¹⁰ copies·ml⁻¹. The positive controls (h) and negative controls (i) illustrated in panel A of FIG. 14 are compared with the experiments comprising one of the materials among a sheet of plastic, paper and wax. The kinetics of panel B of FIG. 14 do not make it possible to determine an influence of the paper or of the wax on the RNA transcription and the DNA amplification. The presence of wax appears to slightly decrease the amplification detection signal. The inhibition illustrated by the curve (l) is weak compared with the amplitude of the signal. The kinetics of panel C of FIG. 14 illustrate the absence of modification of the background signal in the case of negative controls.

FIG. 15 illustrates the influence of a paper substrate on the kinetics of fluorescence signal emitted during an RT-RPA. RT-RPAs are carried out on paper in the kinetics illustrated by FIG. 15, without lyophilization of the reactive compounds. The various reactions are carried out on a square paper substrate comprising four circular areas allowing several simultaneous reactions to be carried out. A mixture of all of the reactive compounds is deposited on the paper substrate. In a positive control reaction, the reactive compounds comprise an RNA target compound 920 at the concentration of 10¹⁰ copies·ml⁻¹. The paper substrates are heated to 40° C. A fluorescence detection makes it possible to monitor the amplification of the DNA transcribed and to distinguish the positive and negative samples. The results of the reactions on paper are compared with identical reactions in the tube. The curve (m) illustrates the fluorescence signal emitted during the RT-RPA in the presence of an RNA target compound 920 (positive control) on paper. The curve (n) illustrates the fluorescence signal emitted during the RT-RPA in the presence of an RNA target compound 920 (positive control) in a tube. The curve (o) illustrates the fluorescence signal emitted during the RT-RPA in the absence of an RNA target compound 920 (negative control) on paper. The curve (p) illustrates the fluorescence signal emitted during the RT-RPA in the absence of an RNA target compound 920 (negative control) in a tube. The curves (m) and (n) exhibit similar profiles and show the good compatibility of carrying out an RT-RPA on a paper substrate.

FIG. 16 illustrates the influence of wax or PDMS barriers, of serum and of lyophilization of the RNA target compound 920 on the kinetics of fluorescence signal emitted during an RT-RPA on paper. The experiments illustrated in FIG. 16 are carried out on a square paper substrate comprising four circular or rectangular areas making it possible to carry out several simultaneous reactions. The reactive compounds are lyophilized on the paper substrate, according to the protocol previously described. They can be kept for several days, at ambient temperature, away from light and moisture. The sample is then deposited, diluted in a buffer or in an aqueous solution, on the substrate, thereby making it possible to rehydrate the substrate. The substrate is then reheated to 40° C. The fluorescence emission is then recorded.

Panel A of FIG. 16 illustrates the effect of a variation in concentration of the RNA reactive compound on the RT-RPA on paper when wax is used to produce the barriers. The curve (neg) illustrates the fluorescence emission for the negative control (no copy of RNA target compound 920), the curve (C₀) illustrates the fluorescence emission for a concentration of 10⁸ copies·ml⁻¹ of RNA target compound 920, the curve (C₁) illustrates the fluorescence emission for a concentration of 10¹⁰ copies·ml⁻¹ of RNA target compound 920, the curve (C₂) illustrates the fluorescence emission for a concentration of 10¹² copies·ml⁻¹ of RNA target compound 920.

Panel B of FIG. 16 illustrates the effect of a variation in concentration of the RNA reactive compound on the RT-RPA on paper when PDMS is used to produce the barriers. The curve (neg) illustrates the fluorescence emission for the negative control (no copy of RNA target compound 920), the curve (C₀) illustrates the fluorescence emission for a concentration of 10⁸ copies·ml⁻¹ of RNA target compound, the curve (C₁) illustrates the fluorescence emission for a concentration of 10¹⁰ copies·ml⁻¹ of RNA target compound 920, the curve (C₂) illustrates the fluorescence emission for a concentration of 10¹² copies·ml⁻¹ of RNA target compound 920.

Panel C of FIG. 16 illustrates the inhibition by human serum of the fluorescence emission during an RT-RPA on paper. The curve (q) illustrates a positive control without serum and the curve (v) illustrates a negative control without serum. The curve (u) illustrates a negative control in the presence of serum diluted five-fold in deionized water. The curve (t) illustrates a positive control in the presence of serum diluted 5-fold. The curve (r) illustrates a positive control in the presence of serum diluted 10-fold. The curve (s) illustrates a positive control in the presence of serum diluted 20-fold.

The panel D of FIG. 16 illustrates the influence of the lyophilization of the RNA target compound 920 on the fluorescence emission of an RT-RPA on paper. The curve (IRNA) illustrates a positive control in the case of the use of lyophilized RNA target compound 920 and the curve (fRNA) illustrates a positive control in the case of the use of fresh RNA target compound 920. The black curve illustrates a negative control.

The results of panel A of FIG. 16 make it possible to conclude that there is a detection threshold for target compound 920 of at most 10⁸ copies·ml⁻¹ (i.e. the detection threshold is at least less than or equal to) in the case of a paper device comprising wax barriers. The study illustrated by panel B of FIG. 16 makes it possible to reach the conclusion of a similar result: the detection threshold for target compound 920 is at most 10⁸ copies·ml⁻¹ (i.e. the detection threshold is at least less than or equal to) in the case of a paper device comprising PDMS barriers. The experiment in which RNA target compounds 920 are diluted in serum makes it possible to measure the size of a possible inhibition in the most realistic sample case. For the most diluted cases (curves (r) and (s)), a slight inhibition is present: the fluorescence signal is weaker than in the case without serum. The curve (t) illustrates that a lower dilution of the serum implies a stronger inhibition.

In one embodiment, a device according to the invention comprises a positive control line and a negative control line. The positive control line and the negative control make it possible to solve prior art problems such as the problem of controlling the good quality of the reactive compounds of the device after storage, and the controlling of external contamination, of the carrier fluid for example. In order to carry out a positive control, the RNA target compound 920 of the device can be lyophilized so as to allow the transportation and storage of the device. Panel D of FIG. 16 illustrates that the same results can be obtained using a fresh or lyophilized target RNA compound 920. The device according to the invention thus makes it possible to carry out a positive and negative control.

FIG. 17 illustrates the storage of a device for amplification by RT-RPA on paper. The positive controls are carried out using an RNA target compound 920 of an Ebola virus at a concentration of 10¹⁰ copies·ml⁻¹.

Panel A of FIG. 17 illustrates several amplifications after different storage times of the device and at ambient temperature. The curve (x) illustrates a positive control after two days of storage of the device, the curve (w) illustrates a positive control after 6 days of storage of the device, the curve (y) illustrates a positive control after 30 days of storage of the device. The black curves illustrate the various negative controls.

Panel B of FIG. 17 illustrates several amplifications carried out after two days of storage, at various temperatures, of devices comprising lyophilized reactive compounds. The curves (z) and (ac) illustrate, respectively, a positive control and a negative control for a prior storage of the device at ambient temperature, the curves (ab) and (ae) illustrate, respectively, a positive control and a negative control for prior storage of the device at 4° C. and the curves (aa) and (ad) illustrate, respectively, a positive control and a negative control for prior storage of the device at −20° C.

Panel C of FIG. 17 illustrates the influence of the lyophilization of all of the reactive compounds and of the carrier fluid on the amplification. The curve (af) illustrates a positive control for a device comprising lyophilized reactive compounds and the amplification of which is carried out using a fresh buffer solution as carrier fluid, and the curve (ag) illustrates a positive control for a device comprising lyophilized reactive compounds and of which the amplification is carried out using a buffer solution having been lyophilized as carrier fluid. The curve (ah) is a negative control.

In the various examples of FIG. 17, the storage of the reactive compounds is tested by producing three devices at the same time. Each device is stored either for two days, or for one week, or for one month, before carrying out an RT-RPA amplification. The results of panel A illustrate the possibility of storing the devices at least for one week without observing any degradation of the amplification results. They also illustrate the possibility of carrying out the detection of a target compound 920 (RNA of an Ebola virus at the concentration of 10¹⁰ copies·ml⁻¹) after one month of storage of the device, despite a slight impairment of the amplification signal. The results of panel B of FIG. 17 illustrate the possibility of storing the devices independently at −20° C., 4° C. and at ambient temperature for detecting a target compound 920. The variation in the positive signal is, in this example, greater in the case of storage at −20° C. Panel C of FIG. 17 illustrates that the use of a fresh buffer solution has no positive consequence on the detection of a target compound 920.

FIG. 18 illustrates the dependence between the amount of reactive compounds deposited on the paper substrate and the geometry of the substrates in question. Panel A of FIG. 18 illustrates kinetics of the fluorescence emission during an RT-RPA on paper for various amounts of reactive compounds deposited on the paper substrate. The curve (ai) corresponds to a condition of depositing prior to the reaction of a volume equivalent to half the standard volume (a standard volume possibly being between 40 μl and 50 μl), the curve (aj) corresponds to a condition of depositing prior to the reaction of a volume equivalent to a quarter of the standard volume and the curve (ak) corresponds to a condition of depositing prior to the reaction of a volume equivalent to an eighth of the standard volume. The black curves correspond to the negative controls for the various conditions of volumes deposited.

Panel B of FIG. 18 illustrates the magnitude of the signal at the end of the reaction as a function of the volume of solution used for the rehydration of the reactive compounds. The experimental points marked by triangles correspond to a concentration of target compound 920 of 10⁸ copies·ml⁻¹, the experimental points marked by squares correspond to a concentration of target compound 920 of 10¹⁰ copies·ml⁻¹ and the experimental points marked by triangles correspond to a concentration of target compound 920 of 10¹² copies·ml⁻¹. The straight black line corresponds to the mean signal of the negative controls and the straight dashed lines correspond to its standard error.

The amount of reactive compounds of the device is suitable for the geometry of the device. The volume deposited comprising the reactive compounds and also the concentration of the solution comprising the reactive compounds have an effect on the detection. A low concentration of reagent tends to increase the value of the detection threshold and to decrease the sensitivity. A high concentration of reactive compounds tends to inhibit the RT-RPA amplification reaction. The reactions corresponding to FIG. 18 are carried out on paper devices of which the barriers are made of PDMS. The experiments described in panel A of FIG. 18 correspond to a device comprising an RNA target compound 920 of an Ebola virus deposited at a concentration of 10¹² copies·ml⁻¹. For a substrate geometry corresponding to an area in the shape of a disk 10 mm in diameter, half the standard volume corresponds to an optimum result. The volume of carrier fluid used for the rehydration of the reactive compounds also modifies the concentration of the reagents during an RT-PCR amplification reaction. A different optimal volume is observed for each concentration of target compound 920. The volume condition V=7.5 μl makes it possible, for the substrate geometry in question, to obtain good detection conditions for 10⁻⁸ copies·ml⁻¹, 10¹⁰ copies·ml⁻¹ and 10¹² copies·ml⁻¹.

FIG. 19 illustrates the flows in a device according to the invention. Panel A of the figure illustrates the flow in the various areas and channels of the device. The photograph at the top of panel A of FIG. 20 illustrates a primary porous substrate opened out during the production of a device. A device described in FIG. 20 makes it possible to carry out a test on a sample capable of containing RNA sequences of an Ebola virus, a negative control and a positive control. Prior to this test, some of the reactive compounds are lyophilized and reactive compounds of said device are arranged in porosities of a primary porous substrate 906 protected by two primary porous substrates 906 located at the ends of the stack 3 required to produce the porous substrate, as described in FIG. 9.

Experiments involving labels make it possible to visualize the flows and to verify the functions of a device. For example, 5 μl of fluorescein are deposited and dried in the area 915 of said device. The photographs at the bottom of panel A of FIG. 20 illustrate a view from above of the device before and after the depositing of a carrier fluid and of a flow of said fluid in all of the areas and channels. The photograph bottom right of panel A of FIG. 20 confirms that the labels deposited are indeed deposited in the three areas of interest. Panel B of FIG. 20 illustrates a similar experiment, with the difference that the labels are deposited before flow in a channel linking the area 915 and the area 950 (shaded part of the photograph at the top of panel B of FIG. 20). The photograph bottom right of panel B of FIG. 20 confirms the transportation of the labels in the area 950 without mixing after the flow.

FIG. 20 illustrates the flows in a device according to the invention. A device described in FIG. 20 makes it possible to carry out three tests of samples capable of containing RNA sequences of an Ebola virus, three negative controls and three positive controls. Prior to this test, some of the reactive compounds are lyophilized and reactive compounds of said device are arranged in porosities of a primary porous substrate 906 and protected by two primary porous substrates 906 located at the ends of the stack 3 required to produce the porous substrate, as described in FIG. 9. Panel A of FIG. 20 comprises two photographs. The photograph on the left illustrates a deposit of fluorescent labels on the area 915. The photograph on the right illustrates the transportation of the fluorescent labels in all of the reading areas 919. Panel B of FIG. 20 comprises two photographs. The photograph on the left illustrates several deposits of fluorescent labels of various colors (Allura Red AC and Brillant Blue G) on areas belonging to two different lines. The photograph on the right illustrates the transportation of the fluorescent labels in all of the reading areas 919. No mixing is noted and the labels are indeed transported in their line up to the reading areas 919. Panel C of FIG. 20 comprises two photographs. The photograph on the left illustrates a deposit of fluorescent labels on the area for depositing a sample 915. The photograph on the right illustrates the transportation of the fluorescent labels in all of the reading areas 919. The fluorescent labels are indeed transported only to the reading areas 919 included in the sample test lines.

FIG. 21 illustrates a detection of the RNA of an Ebola virus. Panel A of FIG. 21 illustrates the fluorescence emissions of the reading areas 919 of a device according to the invention, described for example in FIG. 9. Several RT-RPAs are carried out in each of the lines of the device.

The curve (al) corresponds to the fluorescence of the reading area 919 of the sample test line, the curve (am) corresponds to the fluorescence of the reading area of the positive control line and the curve (an) corresponds to the fluorescence of the reading area of the negative control line. The sample tested corresponds to a sample collected during the Ebola epidemic of 2014 in Macenta. The primers used correspond to the pair of primers of sequence SEQ ID NO.: 5 and SEQ ID NO.: 6. This result shows the possibility of distinguishing a sample comprising RNA of an Ebola virus with a device comprising the primers of the invention.

Panel B of FIG. 21 illustrates the fluorescence emissions of the reading areas 919 of a device according to the invention, described for example in FIG. 9. Several RT-RPAs are carried out in each of the lines of the device. The curve (al) corresponds to the fluorescence of the reading area 919 of the sample test line, the third (am) corresponds to the fluorescence of the reading area of the positive control line and the curve (an) corresponds to the fluorescence of the reading area of the negative control line. The sample tested corresponds to a sample collected during the Ebola epidemic of 2014 in Macenta. The primers used correspond to the pair of primers of sequence SEQ ID NO.: 1 and SEQ ID NO.: 2. This result shows the possibility of distinguishing a sample comprising RNA of an Ebola virus with a device comprising the primers of the invention.

FIG. 22 illustrates the dependence of the conditioning of the reactive compounds and of the target compounds 920 on the kinetics of fluorescence emission of an RT-RPA. RT-RPA reactive compounds are dried (non-lyophilized) on a device such as that described in FIG. 11, for 1 h at 4° C., then stored for two days at −20° C. The reagents contain the primers suitable for an RNA target compound 920 of an Ebola virus (black symbols at a concentration of 10¹⁰ copies/ml). The crosses represent the negative controls for which the reaction medium on paper is rehydrated with water. The circles represent the fluorescence monitoring when the target compound 920 is introduced into the reaction medium.

FIG. 23 illustrates the dependence of the conditioning of the reactive compounds and of the target compounds 920 on the kinetics of fluorescence emission of an RT-RPA. The experimental conditions corresponding to the curves of FIG. 23 are the same as those described in FIG. 22, with the difference that panel A corresponds to a storage of the dried compounds overnight at −20° C., panel B corresponds to a storage of the dried compounds overnight at −4° C. and panel C corresponds to a storage of the dried compounds overnight at 25° C. The distinction between positive and negative is possible when the device is stored at −20° C.

FIG. 24 illustrates tests resulting in the choice of the primers. Paper devices such as that described in panel A of FIG. 12 are prepared for four different primer batches (identified Ref/5.2/4.3/4.4 in table 1 at the end of the description). The curves below illustrate a measurement of fluorescence over time, of each area (the test of the sample corresponds to the gray circles, the negative control corresponds to the black dashes and the positive control corresponds to the black crosses in all of panels A, B, C and D of FIG. 24) for each batch of primers, in response to one and the same test sample.

The batches of primers 4.4 and Ref (see the corresponding sequences in table 1 at the end of the description) are not chosen for the remainder of the experiments because they correspond to a nonspecific signal starting from 10-15 minutes in the negative control area. An additional experiment on another sample makes it possible to choose between the primers 4.3 and 5.2.

FIG. 25 illustrates tests resulting in the choice of the primers. The batch of primers 4.3 appears to be the most efficient for the detection of the test and in the working of the positive controls. This batch of primers is chosen for the remainder of the experiments.

FIG. 26 illustrates the effect of the lyophilization of target compounds 920 and of an RNAse inhibitor on an RT-RPA reaction on paper. On a paper device as described in FIG. 11, the RT-RPA reactive compounds are lyophilized. Fluorescence measurements make it possible to monitor the reaction when the paper is rehydrated and heated. In panels A, B and C of FIG. 26, the square symbols illustrate positive controls, the dashes illustrate negative controls.

The positive control curve of panel A of FIG. 26 illustrates a measurement of fluorescence when the reagents are rehydrated by a solution containing RNA target compounds 920. In panel B of FIG. 26, the RNA target compound 920 is lyophilized at the same time as the RT-RPA reactive compounds. The addition of water makes it possible to rehydrate the reaction medium. The signal is slightly weaker than in the case where the target compound 920 is not lyophilized but is provided in solution at the time of the experiment. The curves of panel C of FIG. 24 illustrate a measurement of fluorescence when the reactive compounds and the RNA target compounds 920 are lyophilized on the paper before the addition of water.

TABLE 1
sequence correspondence

| | | Amplified | | | |
|---|---|---|---|---|---|
| | Region | segment | | | SEQ ID |
| Name | targeted | size | | Sequence | NO: |
| 4.3 | 8661- | 160 | Sense | CTACTGTATTTCATAAGAAGAGAGTTGAACC | 1 |
| | 8820 | | primer | | |
| | | | Antisense | ATTAGTAGGAGTAATTCCCTATCAGTTAA | 2 |
| | | | primer | | |
| | | | Probe | ATATGTCCGACCTTGAAAAAAGGATTTTTG[FAM- | 3 |
| | | | | dT][THG][BHQ₁Dt]GACAGTAGTTTTTGC[3′- | |
| | | | | phosphate] | |
| 4.4 | 8661- | 160 | Sense | CTACTGTATTTCATAAGAAGAGAGTTGAACC | 4 |
| | 8820 | | primer | | |
| | | | Antisense | ATTAGTAGGAGTAATTCCCTATCAGTTAAA | 5 |
| | | | primer | | |
| | | | Probe | AATCCTTTTTTCAAGGTCGGACATATGTC[FAM- | 6 |
| | | | | dT]THF][BHG₁-dT]AGGTGCTGGAGGAAC[3′- | |
| | | | | phosphate] | |
| 5.2 | 17158- | 117 | Sense | CTACTGAGTCCAGTATAGAGTCAGAAATAGTA | 7 |
| | 17274 | | primer | | |
| | | | Antisense | CTGAGTTGTTAAGAATAATCTCAATTTGGT | 8 |
| | | | primer | | |
| | | | Probe | AATGACTCCTAGGATGCTTCTACCTGT[FAM-dT] | 9 |
| | | | | [THF][BHQ₁-dT]GTCAAAATTCCATAA[3′- | |
| | | | | phosphate] | |
| Ref. | 1775- | 169 (Euler, | Sense | GACGACAATCCTGGCCATCAAGATGATGATCC | 10 |
| | 1943 | M et al. | primer | | |
| | | (2013)) | Antisense | CGTCCTCGTCTAGATCGAATAGGACCAAGTC | 11 |
| | | | primer | | |
| | | | Probe | GATGATGGAAGCTAGGCGAATACCAGAG[FAM- | 12 |
| | | | | dT]T[THF]C[BHQ₁-dT]CGGAAAACGGCATG[3′- | |
| | | | | phosphate] | |

1. A device for diagnosis by DNA amplification, comprising:
a porous substrate comprising at least:
an area for depositing a sample, said sample comprising at least one target compound;
a plurality of channels positioned in the thickness of said porous substrate;
an area, referred to as the “diagnostic area”, comprising at least one reactive compound suitable for reacting with a said target compound;
an area for depositing a carrier fluid suitable for being transported by capillarity into all of said areas and said channels:
each of said areas being linked to the others by at least one element chosen from a channel and an area,
a means for locally conditioning the temperature of the diagnostic area.

2. The device as claimed in claim 1, wherein said reactive compounds comprise at least primers, recombinases, polymerases and proteins which bind and maintain a single-stranded DNA, said reactive compounds being arranged in the pores of said areas of said porous substrate.

3. The device as claimed in claim 2, wherein said primers are suitable for detecting the presence of a pathogenic agent by RT-RPA.

4. The device as claimed in claim 3, wherein said pathogenic agent is the Ebola virus.

5. The device as claimed in claim 1, wherein at least one said reactive compound is arranged in or on said porous substrate in lyophilized form.

6. The device as claimed in claim 5, wherein said porous substrate comprises a stack of at least two primary porous substrates, each said primary porous substrate comprising at least one element chosen from a channel, a part of a channel and an area.

7. The device as claimed in claim 6, wherein said stack of said primary porous substrates is obtained by folding.

8. The device as claimed in claim 1, comprising at least one additional area comprising at least one reactive compound of the diagnostic area linked to the area for depositing a carrier fluid by a succession of at least one element chosen from a channel and an area other than an area for depositing a sample.

9. The device as claimed in claim 8, wherein at least one said compound chosen from a reactive compound and a target compound is arranged in or on said porous substrate in lyophilized form.

10. The device as claimed in claim 1, comprising at least one additional area comprising at least one said reactive compound, and at least one target compound, linked to the area for depositing a carrier fluid by a succession of at least one element chosen from a channel and an area other than an area for depositing a sample.

11. The device as claimed in claim 1, comprising a means for locally conditioning the temperature of the area for depositing a sample.

12. The device as claimed in claim 1, wherein said porous substrate comprises at least one sheet of paper.

13. The device as claimed in claim 1, wherein said means for locally conditioning the temperature comprises a conducting electrical circuit placed facing said porous substrate.

14. The device as claimed in claim 13, comprising:
a support layer for the conducting electrical circuit and
a leaktight layer, the leaktight layer being positioned in a manner inserted between the porous substrate and the support layer.

15. The device as claimed in claim 13, wherein said conducting electrical circuit is supported by a sheet of paper distinct from said porous substrate.

16. The device as claimed in claim 1, wherein at least one channel is delimited by regions of said porous substrate which are impregnated with solid wax.

17. The device as claimed in claim 1, wherein at least one channel is formed by a hydrophilic-hydrophobic contrast in the thickness of said porous substrate.

18. The device as claimed in claim 1, wherein said means for locally conditioning the temperature is configured so as to fix the temperature of at least one area between 20° C. and 120° C. and preferentially between 30° C. and 40° C.

19. The device as claimed in claim 1, wherein at least one said reactive compound is suitable for changing color in contact with a said target compound.

20. The device as claimed in claim 1, wherein at least one said reactive compound is suitable for emitting a fluorescence signal on contact with a said target compound.

21. The device as claimed in claim 1, wherein said reactive compounds comprise at least one pair of said primers which is selected from the group consisting of SEQ ID NOS: 1-2; SEQ ID NOS: 4-5; and SEQ ID NOS: 7-8.

22. The device as claimed in claim 1, wherein said reactive compounds comprise at least one nucleotide probe selected from the group consisting of SEQ ID NOS: 3, 6, 9, 13, 14 and 15.

23. The device as claimed in claim 1, wherein said reactive compounds comprise at least one composition of oligonucleotides comprising a pair of primers and a probe, selected from the group consisting of a pair of primers of sequence SEQ ID NOS: 1-2 and a nucleotide probe of sequence SEQ ID NO.: 3; a pair of primers of sequence SEQ ID NOS: 4-5 and a nucleotide probe of sequence SEQ ID NO.: 6; and a pair of primers of sequence SEQ ID NOS: 7-8 and a nucleotide probe of sequence SEQ ID NO.: 9.

24. A process for detecting RNA, comprising at least the steps consisting in:
depositing a said sample on at least one said area for depositing a sample of a said device as claimed in claim 1;
bringing a said carrier fluid into contact with said area for depositing a carrier fluid of said device;
conditioning a stationary temperature of at least one said diagnostic area in such a way as to initiate a reverse transcription of RNA and to amplify a nucleotide sequence selected beforehand, and isothermally;
examining said diagnostic areas to determine the presence or absence of a said target compound.

25. The process as claimed in claim 24, the third step of which comprises a nucleotide sequence amplification by RT-RPA (reverse transcription-recombinase polymerase amplification).

26. A pair of primers which is selected from the group consisting of SEQ ID NOS: 1-2; SEQ ID NOS: 4-5; and SEQ ID NOS: 7-8.

27. A probe selected from the group consisting of SEQ ID NOS: 3, 6, 9, 13, 14 and 15.

28. A composition of oligonucleotides comprising a pair of primers and a probe, selected from the group consisting of a pair of primers of sequence SEQ ID NOS: 1-2 and a nucleotide probe of sequence SEQ ID NO.: 3; a pair of primers of sequence SEQ ID NOS: 4-5 and a nucleotide probe of sequence SEQ ID NO.: 6; and a pair of primers of sequence SEQ ID NOS: 7-8 and a nucleotide probe of sequence SEQ ID NO.: 9.

29. A process for producing a device as claimed in claim 1, comprising at least the steps consisting in:
forming areas and channels in a said porous substrate by heating said porous substrate for at least one minute at a temperature greater than or equal to 100° C. and preferentially greater than or equal to 150° C.;
depositing, on an area of said porous substrate, at least one element chosen from reactive compounds and target compounds diluted in an aqueous solution conditioned at a temperature of between 0° C. and 10° C., and lyophilizing them;
sealing said porous substrate in a plastic sheet.
